SEQUENCE LISTING (13 sequences)

SEQ ID NO: 1 - DNA, 324 bp, Artificial sequence
  a nucleotide sequence of the FKBP12-F36V
atgggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 2 - DNA, 846 bp, Artificial sequence
  a nucleotide sequence of the ??Caspase9
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 3 - DNA, 24 bp, Artificial sequence
  a nucleotide sequence of the Linker
tccggaggaggatccggagtcgac

SEQ ID NO: 4 - DNA, 1032 bp, Artificial sequence
  a nucleotide sequence of the flag gene CD19
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggtga

SEQ ID NO: 5 - DNA, 69 bp, Artificial sequence
  a nucleotide sequence of the T2A
gaattcggcagtggagagggcagaggaagtctgctaacatgcggtgacgtcgaggagaatcctggccca

SEQ ID NO: 6 - DNA, 2295 bp, Artificial sequence
  a nucleotide sequence of the iCaspase9
atgggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatccggaggaggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagaattcggcagtggagagggcagaggaagtctgctaacatgcggtgacgtcgaggagaatcctggcccaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggtga

SEQ ID NO: 7 - DNA, 9624 bp, Artificial sequence
  a nucleotide sequence of the construct
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccaccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcactagttctagaatgggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatccggaggaggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagaattcggcagtggagagggcagaggaagtctgctaacatgcggtgacgtcgaggagaatcctggcccaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggtgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 8 - protein, 480 aa, Artificial sequence
  an amino acid sequence of the ScFv-CD8-4-1BB- CD3??
MALPVTALLLPLALLLHAARPIQMTQTTSSLSASLGDRVTISCRASQDISNFLNWYQQKPDGTVKLLIYYTSRLHSGVPSRFSGSGSGTDFSLTISKLEQEDIATYFCQQGNTLPLTFGAGTKLELKGGGGSGGGGSGGGGSLQQSGPELVKPGASVKISCKASGYTFTDYYMNWVKLSHGKSLEWIGDIVPNNGDTTYNQNFRGKATLTVDKSSSTAYMELRSLTSEDSAVYYCARFSNYVYPFDYWGQGTTITVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 480 aa, Artificial sequence
  an amino acid sequence of the ScFv-CD8-4-1BB- CD3??
MALPVTALLLPLALLLHAARPIQMTQTTSSLSASLGDRVTISCRASQDISNFLNWYQQKPDGTVKLLIYATSRLHSGVPSRFSGSGSGTDFSLTISKLEQEDIATYFCQQGNTLPLTFGAGTKLELKGGGGSGGGGSGGGGSLQQSGPELVKPGASVKISCKASGYTFTDYYMNWVKLSHGKSLEWIGDIVPNNGDTTYNQNFRGKATLTVDKSSSTAYMELRSLTSEDSAVYYCARFSNYVYPFDYWGQGTTITVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1440 bp, Artificial sequence
  an encoding nucleotide sequence of the ScFv- CD8-4-1BB-CD3??
atggccctgcctgtgacagccctgctgctgcctctggctctgctgctgcatgccgctagacccatccagatgacacagactacatcctccctgtctgcctctctgggagacagagtcaccatcagttgcagggcaagtcaggacattagcaattttttaaactggtatcagcagaaaccagatggaactgttaaactcctgatctactacacatcaagattacattctggagtcccatcaaggttcagtggcagtgggtctggaacagatttttctctcaccattagcaaactggagcaagaagatattgccacttacttttgccaacagggtaatacgcttccacttacgttcggcgctgggacaaagttggaacttaaaggtggtggtggttctggcggcggcggctccggaggaggaggatcgctgcaacagtctggacctgagttggtgaagcctggggcttcagtgaagatttcctgcaaggcttctggatacacattcactgactactacatgaattgggtgaagcttagccatggaaagagccttgagtggattggagatattgttcctaacaatggtgatactacttacaaccagaatttcagaggcaaggccacattgactgtagacaagtcctccagcacagcctacatggagctccgcagcctgacatctgaggactctgcagtctattactgtgcaagattcagtaattacgtttacccttttgactactggggccaaggcaccactatcacagtctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 11 - DNA, 1440 bp, Artificial sequence
  an encoding nucleotide sequence of the ScFv- CD8-4-1BB-CD3??
atggccctgcctgtgacagccctgctgctgcctctggctctgctgctgcatgccgctagacccatccagatgacacagactacatcctccctgtctgcctctctgggagacagagtcaccatcagttgcagggcaagtcaggacattagcaattttttaaactggtatcagcagaaaccagatggaactgttaaactcctgatctacgccacatcaagattacattctggagtcccatcaaggttcagtggcagtgggtctggaacagatttttctctcaccattagcaaactggagcaagaagatattgccacttacttttgccaacagggtaatacgcttccacttacgttcggcgctgggacaaagttggaacttaaaggtggtggtggttctggcggcggcggctccggaggaggaggatcgctgcaacagtctggacctgagttggtgaagcctggggcttcagtgaagatttcctgcaaggcttctggatacacattcactgactactacatgaattgggtgaagcttagccatggaaagagccttgagtggattggagatattgttcctaacaatggtgatactacttacaaccagaatttcagaggcaaggccacattgactgtagacaagtcctccagcacagcctacatggagctccgcagcctgacatctgaggactctgcagtctattactgtgcaagattcagtaattacgtttacccttttgactactggggccaaggcaccactatcacagtctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 12 - DNA, 1086 bp, Artificial sequence
  a nucleotide sequence of the EGFRt
tctagaatgttgctgcttgtaacttctctccttctttgcgagttgccccatcctgcgttcctccttattcccaggaaggtatgcaatgggatcggtataggagagttcaaggattccctttctatcaacgctacgaatataaagcacttcaagaactgtacgtccatcagtggagacctgcatatattgccggtggcgttccgaggggacagttttacccacacgccccctctcgacccacaggagctggatatcttgaagaccgtgaaggagataactggctttcttctcattcaggcgtggccggaaaataggacagacttgcacgcctttgaaaacttggaaattatacgagggcggacaaaacaacacggtcaattcagcctggccgttgtatccctcaatatcactagcttgggtctccgaagtctgaaagaaataagtgacggggacgttataatttctgggaacaagaacctctgctacgcaaacacaataaactggaaaaaattgtttggaactagcgggcagaaaactaagatcattagtaacagaggcgagaatagttgcaaagccaccggacaagtgtgccatgcactttgcagccccgagggttgttggggccctgaaccacgggattgcgtgtcatgcagaaacgtctcacgaggtcgcgagtgtgtcgacaaatgtaacctgcttgaaggggagcctcgcgaattcgtagaaaacagcgagtgcattcaatgccacccagagtgtctcccccaggccatgaacatcacctgtacaggacgggggccagataactgtattcaatgcgcacactatatagatggaccacattgtgtgaaaacatgtcccgcaggggtcatgggtgagaacaacacgctcgtttggaaatatgcagatgccgggcatgtatgccacctctgtcacccgaactgcacttatgggtgcactgggcccggcctggaaggatgccccaccaacggacccaagattccctccatagcgaccggaatggttggagccttgcttcttcttctggtagtggcgctcgggatcgggttgttcatgtaaggatcc

SEQ ID NO: 13 - DNA, 8431 bp, Artificial sequence
  a nucleotide sequence of the construct
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccaccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcactagttctagaatgttgctgcttgtaacttctctccttctttgcgagttgccccatcctgcgttcctccttattcccaggaaggtatgcaatgggatcggtataggagagttcaaggattccctttctatcaacgctacgaatataaagcacttcaagaactgtacgtccatcagtggagacctgcatatattgccggtggcgttccgaggggacagttttacccacacgccccctctcgacccacaggagctggatatcttgaagaccgtgaaggagataactggctttcttctcattcaggcgtggccggaaaataggacagacttgcacgcctttgaaaacttggaaattatacgagggcggacaaaacaacacggtcaattcagcctggccgttgtatccctcaatatcactagcttgggtctccgaagtctgaaagaaataagtgacggggacgttataatttctgggaacaagaacctctgctacgcaaacacaataaactggaaaaaattgtttggaactagcgggcagaaaactaagatcattagtaacagaggcgagaatagttgcaaagccaccggacaagtgtgccatgcactttgcagccccgagggttgttggggccctgaaccacgggattgcgtgtcatgcagaaacgtctcacgaggtcgcgagtgtgtcgacaaatgtaacctgcttgaaggggagcctcgcgaattcgtagaaaacagcgagtgcattcaatgccacccagagtgtctcccccaggccatgaacatcacctgtacaggacgggggccagataactgtattcaatgcgcacactatatagatggaccacattgtgtgaaaacatgtcccgcaggggtcatgggtgagaacaacacgctcgtttggaaatatgcagatgccgggcatgtatgccacctctgtcacccgaactgcacttatgggtgcactgggcccggcctggaaggatgccccaccaacggacccaagattccctccatagcgaccggaatggttggagccttgcttcttcttctggtagtggcgctcgggatcgggttgttcatgtaaggatccaccggtcgccaccagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

(19) United States
(12) Patent Application Publication
(10) Pub. No.: US 2021/0401957 A1
(43) Pub. Date: Dec. 30, 2021

(54) ROBO1 CAR-NK CELL CARRYING SUICIDE GENE, PREPARATION METHOD AND APPLICATION THEREOF

(71) Applicant: Asclepius (Suzhou) Technology Company Group Co., Ltd., Suzhou (CN)

(72) Inventors: Kunkun HAN, Suzhou (CN); Jianmin FU, Suzhou (CN); Min ZHOU, Suzhou (CN); Fanjun ZENG, Suzhou (CN); Qiang WANG, Suzhou (CN); Huashun LI, Suzhou (CN)

(21) Appl. No.: 17/474,927

(22) Filed: Sep. 14, 2021

Related U.S. Application Data
(63) Continuation of application No. PCT/CN2020/077582, filed on Mar. 3, 2020.

(30) Foreign Application Priority Data
Mar. 15, 2019 (CN) 201910196531.5

(51) Int. Cl. A61K 39/00; C12N 15/86; C12N 9/64; C12N 15/62; C07K 14/725; C07K 16/28; C07K 14/705; A61P 35/00

(52) U.S. Cl. CPC A61K 39/001111; A61K 2039/812; C12N 9/6472; C12Y 304/22062; C12N 15/62; C07K 14/7051; C07K 16/2803; C07K 14/70517; C07K 14/70578; A61P 35/00; C12N 2740/15043; C07K 2319/02; C07K 2319/03; C07K 2317/55; C07K 2317/622; C12N 15/86

(57) ABSTRACT

Provided is a ROBO1 CAR-NK cell carrying a suicide gene, and a preparation method and application thereof. In order to increase the safety and controllability of a CAR-NK therapy, on the basis of a present ROBO1 CAR-NK cell, a suicide gene switch element is integrated into a genome by means of a lentiviral transfection technology to form a CAR-NK carrying a suicide gene. By adding the suicide gene, the CAR-NK cell can be better controlled, and the clinical safety is further improved.

### CROSS-REFERENCE

The present application is a continuation of International Application No. PCT/CN2020/077582, filed on Mar. 3, 2020, which claims priority to Chinese Patent Application No. 201910196531.5, filed on Mar. 15, 2019. All of the aforementioned applications are hereby incorporated by reference in their entireties.

### TECHNICAL FIELD

The present invention belongs to the technical field of biopharmaceuticals, and relates to a NK cell and a preparation method and application thereof, and particularly to a ROBO1 CAR-NK cell carrying a suicide gene and a preparation method and application thereof.

### BACKGROUND

In recent years, CAR-T cell-based immunotherapy has received extensive attention in the industry. As a “living” medicament, CAR-T therapy is quite different from traditional medicament. First, this therapy needs to isolate T cells from patients, and uses chimeric antigen receptors (CAR) to modify the T cells in vitro so that the T cells can specifically recognize cancer cells, and then the modified T cells are expanded and reinfused into the patients. For example, the successful application of CD19 CAR-T cells in patients with CD19 positive malignant tumors proves the feasibility of this method for cancer immunotherapy (Grupp et al., 2013). Moreover, CAR-T cells targeting a plurality of different tumor antigens are being actively developed clinically (Kalos et al., 2013).

Although CAR-T therapy has shown great potential, it has obvious limitations. First, the cells have to be isolated from the body (this process is time-consuming and costly). Moreover, because the T cells are modified for specific patients, some patients may not be able to collect the T cells or have insufficient time to wait for the preparation process of the T cells. Although CAR-T is developing towards universal CAR-T, it actually increases clinical risks and operational difficulties. Moreover, because the T cells are modified for specific patients, some patients may not be able to collect the T cells or have insufficient time to wait for the preparation process of the T cells. In addition, faced with the high cost of CAR-T (referring to two listed companies, Novartis and Kite), these limitations may cause some patients who are expected to benefit to fail to accept the CAR-T immunotherapy. In addition, the CAR-T therapy still has serious toxicity or side effects, including cytokine release syndrome CRS, neurotoxicity, and the like.

Natural killer (NK) cells are an important effector cell type for adoptive cancer immunotherapy. Similar to the T cells, the NK cells may be modified to express chimeric antigen receptors (CARs) to enhance anti-tumor activity. The NK cells play an important role in cancer immune surveillance and represent an important effector cell type for adoptive cancer immunotherapy. Compared with the T cells, the NK cells do not need to activate and recognize peptide antigens presented by complex MHC molecules in advance. On the contrary, the NK cells can show killing activity under appropriate stimulation by coupling coded cell surface receptors with a CD3 molecules. Therefore, the CAR-NK therapy is expected to become an important development orientation of tumor cell therapy.

By modifying the NK92 cells and loading CAR, the targeting ability of the cells is greatly increased. Meanwhile, a suicide gene switch element is installed to further improve the safety and controllability of the CAR-NK cells. Moreover, pre-clinical experiments also prove that our CAR-NK cells do not have obvious toxic and side effects. However, in order to further increase the safety and effectiveness of the CAR-NK therapy, further research and improvement are needed.

### SUMMARY

In view of the above technical problems, one object of the present invention is to provide a nucleotide sequence carrying a suicide gene, another object of the present invention is to provide a ROBO1 CAR-NK cell carrying a suicide gene, and one another object of the present invention is to provide an application of the ROBO1 CAR-NK cell carrying the suicide gene, which can better control the CAR-NK cell by adding a suicide gene switch element on the basis of the ROBO1 CAR-NK cell, thus further increasing the clinical safety.

The present invention provides a nucleotide sequence carrying a suicide gene, including a gene encoding a chimeric antigen receptor, and further including a suicide inducing gene, wherein the chimeric antigen receptor includes an antigen binding domain, a transmembrane domain and a costimulatory signal transduction region, the antigen binding domain is capable of specifically binding to a tumor specific antigen, and activating a NK cell through the transmembrane domain and the costimulatory signal transduction region, and the tumor specific antigen is ROBO1.

In a preferred embodiment of the present invention, the suicide inducing gene is iCaspase9, EGFRt, CD20, rapamycin and/or RQR8.

In a preferred embodiment of the present invention, the suicide inducing gene is iCaspase9, the iCaspase9 includes FKBP12-F36V and ΔCaspase9, a nucleotide sequence of the FKBP12-F36V is shown in SEQ ID NO: 1, and a nucleotide sequence of the ΔCaspase9 is shown in SEQ ID NO: 2.

In a preferred embodiment of the present invention, the FKBP12-F36V and the ΔCaspase9 are connected through a Linker, and a nucleotide sequence of the Linker is shown in SEQ ID NO: 3. The design of a Linker sequence is one of the key technologies for the success of a gene fusion technology. Different target genes are connected by appropriate Linker sequences, making the target genes express as a single peptide chain in an appropriate organism, wherein an amino acid which plays a connecting role is called Linker. The Linker does not affect respective functions of target proteins, and enables a fusion protein to form a correct spatial structure and gives better play to biological activity.

In a preferred embodiment of the present invention, the suicide gene iCaspase9 is further provided with a flag gene, and the flag gene is preferably CD19, Myc, Flag, HA or His, and the flag gene is tested to prove whether the target protein is correctly expressed.

In a preferred embodiment of the present invention, the flag gene may be CD19, Myc, Flag, HA, or His, and is preferably CD19; the suicide gene iCaspase9 is further provided with a splicing gene, and the splicing gene is T2A or P2A, and preferably T2A; a nucleotide sequence of the flag gene CD19 is shown in SEQ ID NO: 4, and a nucleotide sequence of the T2A is shown in SEQ ID NO: 5. Through the flag gene CD19, the Caspase9 protein expressed in cells can be recognized. In addition, a flag of the fusion protein after mRNA translation can be successfully spliced from iCaspase9 under the action of the splicing gene. CD19 is a truncated CD19, including an extracellular segment and a transmembrane region of CD19 and an intracellular truncated sequence (excluding an intracellular signal transduction region), which is only used as a label for testing the suicide genes, and has no other functions. Moreover, as a test label of the suicide genes, CD19 has also shown good test results in clinical patients, and has higher safety than other labels. Two genes are connected into one ORF by T2A polypeptide, and become one fusion protein after mRNA translation. However, this fusion protein will be spliced into two proteins by a protease recognizing 2A. The two proteins are expressed separately, and the functions thereof do not affect each other. T2A has the advantage of very short sequence, which basically has no influence on protein functions.

In a preferred embodiment of the present invention, the nucleotide sequence of the iCaspase9 is shown in SEQ ID NO: 6.

In a preferred embodiment of the present invention, the suicide inducing gene is EGFRt, and the EGFRt includes an extracellular domain III, an extracellular domain IV and a transmembrane region of EGFR.

In a preferred embodiment of the present invention, a nucleotide sequence of the EGFRt is shown in SEQ ID NO: 12.

In a preferred embodiment of the present invention, the antigen binding domain is capable of specifically binding to one or more of Ig1, Ig2, Ig3, Ig4, Ig5, FN1, FN2 and FN3 domains of the tumor specific antigen ROBO1.

In a preferred embodiment of the present invention, the antigen binding domain is capable of specifically binding to the FN3 domain of the tumor specific antigen ROBO1.

In a preferred embodiment of the present invention, the antigen binding domain is an antibody or an antigen binding fragment thereof specifically binding to the FN3 domain of ROBO1, and the antigen binding fragment is Fab or ScFV.

In a preferred embodiment of the present invention, the transmembrane domain is selected from one or more of CD28, CD3c, CD45, CD4, CD5, CD8, CD9, CD16, CD22, CD33, CD37, CD134, CD137, ICOS and CD154; and preferably, the transmembrane domain is a CD8 transmembrane domain; and/or, the costimulatory signal transduction region contains an intracellular domain of a costimulatory molecule, and the costimulatory molecule is selected from one or more of CD3ζ, CD3γ, CD3δ, CD3ε, CD5, CD22, CD79a, CD79b, CD66d, CD2, CD4, CD5, CD28, CD13ζ, CD137, ICOS, CD154, 4-1BB and OX40; and preferably, the costimulatory signal transduction region contains intracellular domains of 4-1BB and CD3.

In a preferred embodiment of the present invention, the chimeric antigen receptor includes a fusion protein with a structure of ScFV-CD8-4-1BB-CD3ζ, and the ScFv is capable of specifically binding to the FN3 domain of the tumor specific antigen ROBO1.

In a preferred embodiment of the present invention, an amino acid sequence of the fusion protein ScFv-CD8-4-1BB-CD3ζ is shown in SEQ ID NO: 8 or SEQ ID NO: 9.

In a preferred embodiment of the present invention, an encoding nucleotide sequence of the fusion protein ScFv-CD8-4-1BB-CD3ζ is shown in SEQ ID NO: 10 or SEQ ID NO: 11.

The present invention further provides a construct carrying a suicide gene, including the nucleotide sequence mentioned above.

In a preferred embodiment of the present invention, the construct is a lentiviral construct; and preferably, when the suicide gene is iCaspase9, a nucleotide sequence of the construct is shown in SEQ ID NO: 7; or

when the suicide gene is EGFRt, the nucleotide sequence of the construct is shown in SEQ ID NO: 13.

The present invention further provides a chimeric antigen receptor carrying a suicide gene, encoded by the above-mentioned nucleotide sequence.

The present invention further provides a ROBO1 CAR-NK cell carrying a suicide gene, wherein the ROBO1 CAR-NK cell expresses the chimeric antigen receptor.

In a preferred embodiment of the present invention, the ROBO1 CAR-NK cell carrying the suicide gene is capable of effectively destroying or killing a lung cancer cell, a pancreatic cancer cell, a hepatoma cell, a breast cancer cell, a colon cancer cell, a prostate cancer cell or a gastric cancer cell.

The present invention further provides a preparation method of the above-mentioned ROBO1 CAR-NK cell carrying the suicide gene, including the following steps of:

(1) synthesizing and amplifying the suicide gene in the above-mentioned nucleotide sequence, and cloning the nucleotide sequence into a lentiviral expression vector to obtain a lentiviral vector carrying the suicide gene; and

(2) packaging a lentivirus through a lentivirus packaging cell line and a three-plasmid system including the lentivirus vector carrying the suicide gene obtained in step (1) to obtain a lentivirus carrying the suicide gene, and then infecting the ROBO1 CAR-NK cell with the lentiviral carrying the suicide gene to integrate the suicide gene into a genome of the ROBO1 CAR-NK cell to obtain the ROBO1 CAR-NK cell carrying the suicide gene.

In a preferred embodiment of the present invention, the ROBO1 CAR-NK cell is prepared through a method including the following steps of:

a. synthesizing and amplifying a nucleotide sequence encoding the chimeric antigen receptor, and cloning the nucleotide sequence into a lentiviral expression vector; and preferably, synthesizing the above-mentioned encoding nucleotide sequence of the fusion protein ScFv-CD8-4-1BB-CD3ζ to obtain a lentiviral vector containing the encoding nucleotide sequence of the fusion protein ScFv-CD8-4-1BB-CD3ζ;

b. packaging through a plasmid required by lentiviral packaging and the lentivirus expression vector obtained in step a in a packaging cell line to prepare a lentivirus; and

c. infecting a NK cell with the lentivirus obtained in step b to obtain the ROBO1 CAR-NK cell.

The present invention further provides a pharmaceutical composition including the above-mentioned ROBO1 CAR-NK cell carrying the suicide gene or the ROBO1 CAR-NK cell carrying the suicide gene prepared by the above-mentioned method; and preferably, the pharmaceutical composition further including an inducer, wherein when the suicide gene is iCaspase9, the inducer is preferably AP1903 or AP20187; and a concentration of the small molecule chemical inducer is 0 nM to 50 nM; or

when the suicide gene is EGFRt, the inducer is preferably cetuximab with an action concentration of 1 μg/ml.

In a preferred embodiment of the present invention, when the ROBO1 CAR-NK cell carrying the suicide gene is used for killing a tumor, an effector-to-target ratio of the ROBO1 CAR-NK cell carrying the suicide gene to a tumour cell is 0.5:1 to 5:1, and the effector-to-target ratio is preferably 0.5:1 to 1:1.

In a preferred embodiment of the present invention, the pharmaceutical composition further includes pharmaceutically acceptable salts, carriers, excipients and adjuvants.

According to some general methods in the technical field of the present invention, the medicament of the present invention may be prepared into various pharmaceutically acceptable dosage forms, such as tablets, capsules, oral liquids, lozenges, injections, ointments, granules or various sustained and controlled release preparations, or the like, and preferably injection, which may be administered by subcutaneous injection.

The carriers of the medicament of the present invention may be common types available in the pharmaceutical field, including adhesives, lubricants, disintegrants, cosolvents, diluents, stabilizers, stabilizers or matrix, and the like.

The present invention further provides an application of the above-mentioned ROBO1 CAR-NK cell carrying the suicide gene or the ROBO1 CAR-NK cell carrying the suicide gene prepared by the above-mentioned preparation method in the preparation of a medicament for treating and/or preventing a cancer; and preferably, the cancer is a tumor with high expression of ROBO1 and related diseases.

In a preferred embodiment of the present invention, the cancer is liver cancer, breast cancer, colon cancer, pancreatic cancer, prostate cancer, gastric cancer, or lung cancer.

In a preferred embodiment of the present invention, the medicament is a medicament in an intratumoral administration form, such as a medicament in an intratumoral injection form or a medicament in an intravenous infusion form.

The present invention further provides a method for treating and/or preventing a cancer by using the above-mentioned ROBO1 CAR-NK cell carrying the suicide gene, or the ROBO1 CAR-NK cell carrying the suicide gene prepared, or the pharmaceutical composition, wherein the method includes administering an effective amount of the pharmaceutical composition containing the ROBO1 CAR-NK cell carrying the suicide gene into a patient.

In a preferred embodiment of the present invention, the cancer is a tumor with high expression of ROBO1 and related diseases.

In a preferred embodiment of the present invention, the cancer is liver cancer, breast cancer, colorectal cancer, pancreatic cancer, prostate cancer, gastric cancer, or lung cancer; and preferably, the cancer is breast cancer, colorectal cancer or liver cancer.

In a preferred embodiment of the present invention, a dosage of the ROBO1 CAR-NK cell carrying the suicide gene is 0.5×10⁹ cells/times to 5×10⁹ cells/times.

In a preferred embodiment of the present invention, an administration mode of the ROBO1 CAR-NK cell carrying the suicide gene is intratumoral injection, intravenous injection, intrathoracic injection or local intervention.

In a preferred embodiment of the present invention, the administration mode of the ROBO1 CAR-NK cell carrying the suicide gene is intravenous injection.

The present invention has the beneficial effects that: firstly, the ROBO1 CAR-NK cells may be used as a therapeutic medicament for tumor diseases, used for treating tumors with high expression of ROBO1 molecules, and have no undesirable phenomena such as cytokine storm and the like, thus providing a new treatment method for tumors ineffective by traditional surgery, chemotherapy and radiotherapy; moreover, the targeting ability of the cells is greatly increased by modifying the NK92 cells and loading CAR, and the target in CAR is an anti-tumor target ROBO1, which greatly improves the anti-tumor performance thereof while improving the targeting ability; in addition, upon preclinical experiments, it is found that the toxic and side effects of the ROBO1CAR-NK currently used by us are smaller than those of CAR-T, and the killing effect is better; that is to say, the targeting ability of the ROBO1 CAR-NK cell constructed by the present invention is improved by firstly modifying the NK92 cells and loading CAR; secondly, the NK92 cells are isolated from a peripheral blood of a lymphoma patient and a cell line is successfully established; although the NK92 cells can identify target cells without WIC limitation, can kill tumor cells without pre-sensitization, and can also produce a series of cytokines, so as to regulate the acquired immunity of the body, the ROBO1 CAR-NK also has the function of regulating secondary immunity, but still has some tumor formation risks after being used in a human body.

Therefore, in order to increase the safety and controllability of the CAR-NK therapy, on the basis of the present ROBO1 CAR-NK cell, the suicide gene switch element is integrated into the genome by means of the lentiviral transfection technology to form the CAR-NK carrying the suicide genes with different mechanisms of action. When the small molecule chemical inducer, such as AP1903, is added in vitro, the small molecule chemical inducer can combine with the domain on FKBP12-F36V in the suicide gene, and then form dimeric iCaspase9, thus activating Caspases 3, 6 and 7 and inducing apoptosis; when the inducer cetuximab is added in vitro, the ROBO1 CAR-NK carrying the suicide gene is under the action of killer/effector cells (NK92-CD16, i.e., the NK92 cell line with high expression of CD16 molecule) or PBMC, the cetuximab is combined with EGFRt, and the Fc segment thereof is combined with FcR on the surface of NK92-CD16 or PBMC, thereby initiating antibody-dependent cell-mediated cytotoxicity (ADCC), mediating the killer/effector cells to directly kill the target cells, thus better controlling the CAR-NK cells, further increasing clinical safety and controllability, and promoting the development of the CAR-NK therapy.

### BRIEF DESCRIPTION OF THE DRAWINGS

The present invention can be more clearly understood from the following detailed description with reference to the accompanying drawings, wherein:

FIG. 1 is a schematic structural diagram of a lentiviral plasmid vector pRRLSIN-ScFv (anti ROBO1-FN3) provided in Example 1 of the present invention;

FIG. 2 is a schematic structural diagram of a lentiviral plasmid vector (1942-3-IC9) carrying a suicide gene iCaspase9 in Example 4 of the present invention;

FIGS. 3a-3b show results of CAR positive rates of ROBO1 CAR-NK cells by flow cytometry provided in Example 3 of the present invention;

FIGS. 4a-4b show results of CD56 molecule positive rates of the ROBO1 CAR-NK cells using flow cytometry provided in Example 3 of the present invention;

FIG. 5 is a diagram showing iCaspase9 and ROBO1 positive rates of the ROBO1 CAR-NK cells carrying a suicide gene using flow cytometry provided in Example 4 of the present invention, wherein PE is a channel for testing Flag, and the positive rate in the Flag-PE channel indicates the result of the positive rate of ROBO1 CAR, APC is a channel for testing CD19, the positive rate in the APC-CD19 channel indicates the result of the positive rate of iCaspase9, Flag/CD19 indicates the double positive result tested, and the positive rate of the double positive indicates the result of the positive rate of simultaneous expression of iCaspase9 and ROBO1;

FIG. 6 shows a test result of an mRNA level of the ROBO1 CAR-NK cell carrying the suicide gene provided in Example 4 of the present invention by RT-PCR, wherein 1 is a NK-92 cell, 2 is a ROBO1 CAR-NK cell, 3 is a ROBO1-iCaspase9 cell, and iCas9 in the figure indicates iCaspase9;

FIG. 7 shows a whole genome sequencing result of a ROBO1 CAR-NK cell carrying a suicide gene in Example 5 of the present invention, and the result is the analyzed information of inserted genome sites;

FIG. 8 shows a stability test result of a ROBO1 CAR-NK cell carrying a suicide gene in Example 6 of the present invention;

FIG. 9 shows a result of inducing apoptosis of the ROBO1 CAR-NK cell carrying the suicide gene in Example 6 of the present invention with AP1903;

FIG. 10a shows a result of a positive rate of the ROBO1 CAR-NK cell ATCG427B-1F7 carrying the suicide gene by flow cytometry in Example 6 of the present invention;

FIG. 10b shows a result of a positive rate of the ROBO1 CAR-NK cell ATCG427B-2D5 carrying the suicide gene by flow cytometry in Example 6 of the present invention;

FIG. 11 shows a result of inducing apoptosis of a ROBO1 CAR-NK cell carrying a suicide gene in Example 7 of the present invention with different concentrations of AP1903;

FIG. 12 shows a result of counting a cell survival rate after the cells treated with different concentrations of AP1903 for 2 hours, 4 hours and 24 hours are continuously cultured in fresh media for three days in Example 7 of the present invention;

FIG. 13 is a result of culturing an experimental group treated for 4 hours in Example 7 of the present invention in a fresh medium for 24 hours.

FIG. 14a shows test results of ROBO1 protein expression in different breast cancer cells in Example 8 of the present invention, wherein HCC1187, HCC1937, HCC38, MDAMB-231, MDAMB-453 and MDAMB-468 are triple-negative breast cancer cell lines;

FIG. 14b shows a result of a ratio of an optical density of ROBO1 to an optical density of GAPDH determined and tested in FIG. 14a;

FIG. 15 shows a killing result of the ROBO1 CAR-NK cell carrying a suicide gene in Example 9 of the present invention used for breast cancer cells;

FIGS. 16a-c show killing results of the ROBO1 CAR-NK cell carrying the suicide gene in Example 9 of the present invention used for different cancer cells;

FIG. 17a shows a test result of ROBO1 expression in a normal breast cell and PBMC in Example 9 of the present invention;

FIG. 17b shows an experimental result of killing the normal breast cell and PBMC by the ROBO1 CAR-NK cell carrying the suicide gene in Example 9 of the present invention;

FIG. 18 shows a killing result of PBMC on the ROBO1 CAR-NK cell carrying the suicide gene in Example 9 of the present invention;

FIG. 19 is a schematic structural diagram of a lentiviral plasmid vector 1942-3-hEGFRt provided in Example 10 of the present invention;

FIG. 20 is a schematic structure diagram of EFGRt in Example 10 of the present invention;

FIG. 21a shows a result of a positive rate of the ROBO1 CAR-NK cell ROBO1-EGFRt-3C10-2D10 carrying the suicide gene using flow cytometry in Example 10 of the present invention;

FIG. 21b shows a result of a positive rate of the ROBO1 CAR-NK cell ROBO1-EGFRt-3C10-1F11 carrying the suicide gene using flow cytometry in Example 10 of the present invention;

FIG. 22 shows test results of ROBO1 protein expression levels in different types of cancer cells in Example 11 of the present invention;

FIG. 23a is an experimental diagram showing killing results of the ROBO1 CAR-NK cell carrying the suicide gene in Example 11 of the present invention for killing experiments;

FIG. 23b is an experimental diagram for determining specific killing results of the ROBO1 CAR-NK cell carrying the suicide gene in Example 11 of the present invention for killing experiments by using a ROBO1-overexpressed monoclonal cell strain (HCT116-ROBO1) model;

FIG. 23c is an experimental diagram for determining specific killing results of the ROBO1 CAR-NK cell carrying the suicide gene in Example 11 of the present invention for killing experiments by using a ROBO1-overexpressed monoclonal cell strain (MDAMB-231-ROBO1) model;

FIG. 24 is an experimental result diagram of evaluating effectiveness of a suicide switch in the ROBO1-EGFRt-CAR-NK cell carrying a suicide gene by a NK92-CD16 (high affinity) effector cell constructed in vitro in Example 12 of the present invention; and

FIG. 25 is an experimental result diagram of evaluating the effectiveness of the suicide switch in the ROBO1-EGFRt-CAR-NK cell carrying the suicide gene by a PBMC effector cell in Example 12 of the present invention;

wherein, ATCG427A-2h, ATCG427A-4h and ATCG427A-24h respectively represent results of inducing the ATCG427A with AP1903 for 2 hours, 4 hours and 24 hours; ATCG427B-1F7-2h, ATCG427B-1F7-4h and ATCG427B-1F7-24h respectively represent results of inducing the ATCG427B-1F7 with AP1903 for 2 hours, 4 hours and 24 hours respectively; while ATCG427B-2D5-2h, ATCG427B-2D5-4h and ATCG427B-2D5-24h respectively represent results of inducing the ATCG427B-2D5 with AP1903 for 2 hours, 4 hours and 24 hours; and

T47D-2h, HCC1187-2h, MCF-2h, HepG2-2h, Huh7-2h, HCT116-2h, HCT116-Flag.ROBO1.Full-1C11-2h, HCT116-Flag.ROBO1.Full-1D11-2h, MDAMB-231-2h, MDAMB-231-Flag.ROBO1.Full-1C11-2h, and MDAMB-231-Flag.ROBO1.Full-1D11-2h all indicate the corresponding cancer cells that are induced to kill for 2 hours, wherein HCT116-Flag.ROBO1.Full-1C11-2h and HCT116-Flag.ROBO1.Full-1D11-2h respectively indicate of being induced to kill for 2 hours by different clones of the ROBO1-overexpressed HCT116-ROBO1 model; while MDAMB-231-Flag.ROBO1.Full-1C11-2h and MDAMB-231-Flag.ROBO1.Full-1D11-2h respectively represent of being induced to kill for 2 hours by different clones of the ROBO1-overexpressed MDAMB-231-ROBO1 model.

### DETAILED DESCRIPTION OF EXAMPLES

Various exemplary examples of the present invention will now be described in detail with reference to the accompanying drawings. It should be noted that the parameter values of the methods and steps illustrated in these examples do not limit the scope of the present invention unless otherwise specified.

The following description of at least one exemplary example is merely illustrative in nature and in no way serves as any limitation on the present invention and application or use thereof.

Techniques and methods known to those of ordinary skills in the related arts may not be discussed in detail, but under appropriate circumstances, such techniques and methods should be regarded as a part of the specification.

Unless otherwise specified, “NK cells” herein include “peripheral blood NK cells, NK92 cells and other NK cells”.

Unless otherwise specified, the “NK-92 cells” and “NK cells” herein share the same meaning.

Unless otherwise specified, “ROBO1 CAR-NK” herein refers to “a chimeric antigen receptor cell targeting ROBO1, especially an enhanced CAR-T cell and CAR-NK cell targeting ROBO1”, and the English names appearing herein are not case-sensitive, such as ROBO1, Robo1, robo1, and the like, which share the same meaning. Robo1 CAR-NK and Robo1-CAR NK share the same meaning, the specific preparation processes of which are referred to the following examples.

Unless otherwise specified, “ATCG427A” herein refers to “ROBO1 CAR-NK cell” or “ROBO1MCAR-NK”.

Unless otherwise specified, “iCaspase9” and “iCas9” herein both refer to “suicide inducing gene Caspase9”.

Unless otherwise specified, “ROBO1-iCaspase9 CAR-NK cell” herein refers to “ROBO1 CAR-NK cell carrying suicide gene”.

Unless otherwise specified, “ATCG427B” herein refers to “ROBO1 CAR-NK cell carrying suicide gene iCaspase9, i.e., ROBO1-iCaspase9 CAR-NK cell”.

Unless otherwise specified, “FKBP12” herein refers to “FKBP12-F36V”.

Unless otherwise specified, “CMV” herein refers to “CMV promoter”.

Unless otherwise specified, “ATCG427E” herein refers to “ROBO1 CAR-NK cell carrying suicide gene EGFRt, i.e., ROBO1-EGFRt CAR-NK cell”.

Unless otherwise specified, “ATCG427A-KO” herein refers to “knocking out the gene sequence of a ROBO1-scFV target in CAR in the form of Crisp Cas9 gene editing, keeping other signal transduction sequences, and then knocking the CAR into a NK92 cell, that is, the NK cell in which the CAR sequence without the ROBO1 target is knocked on the same genome insertion site as CAR in 427A”.

Unless otherwise specified, “NK” described in the present invention is a human normal NK cell or NKT cell, or NK cell line, including NK92 cell, YT cell, NKL cell, HANK-1 cell, NK-YS cell, KHYG-1 cell, SNK-6 cell, IMC-1 cell, and the like”. In the specific embodiment of the present invention, NK-92 cell is taken as an example.

Unless otherwise specified, “vector” herein is a physical composition, which includes isolated nucleic acids, and can be used to transfer isolated nucleic acids into a cell. Many vectors are known in this field, including, but not limited to, linear polynucleotides, and the polynucleotides related to ions or amphiphilic compounds, plasmids and viruses. Therefore, the term “vector” includes autonomously replicating plasmids or viruses. The term should also be interpreted as including non-plasmid and non-viral compounds that facilitate transferring nucleic acids into cells, such as polylysine compounds, liposomes and the like. Examples of viral vectors include, but not limited to, adenovirus vectors, adeno-associated virus vectors, retroviral vectors, and so on.

Unless otherwise specified, “antibody” herein includes a whole antibody and any antigen binding fragment (i.e., “antigen binding part”) or single chain thereof.

Unless otherwise specified, all reagents herein are analytical-grade pure and may be purchased from regular channels.

Material sources in the following examples:

NK-92 cell (CC® CRL-2407), breast cancer cells BT474, T47D, HCC1187, HCC1937, MCF7, MDAMB-231, MDAMB-453, MDAMB-468 and ZR-75-1, lung cancer cells A549 and H1299, hepatoma cells Huh7, SMMC7721 and HEPG2, pancreatic cancer cells BxPC3, PANC1 and Capan-2, and Lenti-X-293T cell, and colorectal cancer cells such as HT-29, LoVo and HCT116 were all purchased from the Cell Resource Center of Shanghai Institutes for Biological Sciences, Chinese Academy of Sciences.

### Example 1. Preparation of Lentiviral Vector

A sequence of a ScFV (Anti ROBO1-FN3)-CD8(CD8TM)-4-1BB-CD3ζ fusion gene (with an amino acid sequence shown in SEQ ID NO: 8 and a gene sequence shown in SEQ ID NO: 10) and a sequence of a mutant ScFV (Anti ROBO1-FN3)-CD8(CD8TM)-4-1BB-CD3ζ fusion gene (with an amino acid sequence shown in SEQ ID NO: 9, a gene sequence shown in SEQ ID NO: 11, and a mutation site of GCC or GCG) were synthesized separately. The ScFV (Anti ROBO1-FN3)-CD8TM-4-1BB-CD3ζ fusion gene was taken as an example to illustrate the preparation process of ROBO1 CAR-NK cells, while the preparation process of ROBO1M CAR-NK cells using the mutant ScFV (Anti ROBO1-FN3)-CD8TM-4-1BB-CD3ζ fusion gene was identical.

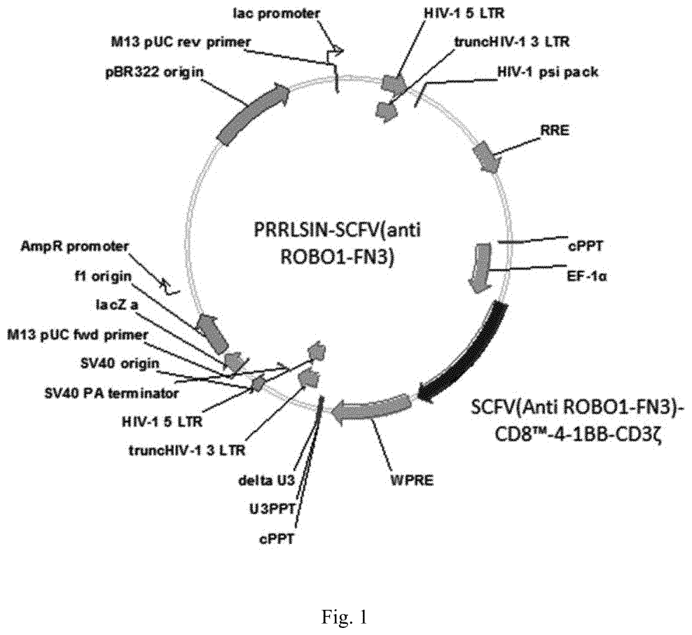

The sequence of the ScFV (Anti ROBO1-FN3)-CD8-4-1BB-CD3ζ fusion gene was transformed and ligated into a pRRLSIN vector by enzyme digestion, wherein the upstream of the gene was EP-la promoter. The vector was transformed into Stb13 Escherichia coli strain, which was screened by ampicillin to obtain positive clones. Then plasmid was extracted and the clone was identified by enzyme digestion, so that a pRRLSIN-ScFv (anti ROBO1-FN3) lentiviral transfection vector was obtained (as shown in FIG. 1).

### Example 2. Preparation of Lentiviral

(1) 24 hours before transfection, Lenti-X-293T cells were inoculated into 15 cm petri dishes at about 8×10⁶ per dish. It was ensured that the convergence degree of the cells was about 80% and the cells were evenly distributed in the petri dishes during the transfection.

(2) Preparation of solution A and solution B

Solution A: 6.25 mL of 2×HEPES buffer (the effect was better when the quantity was a package of 5 dishes together)

Solution B was a mixture by adding the following plasmids respectively: 112.5 μg of pRRLSIN-ScFv (anti ROBO1-FN3) (target plasmid); 39.5 μg of pMD2.G (VSV-G envelop); 73 μg of pCMVR8.74 (gag, pol, tat and rev); and 625 μl of 12M calcium ion solution. The total volume of the solution B was 6.25 mL.

Solution B was mixed completely, and dropwise added into solution A while gently swirling solution A, so that a mixture of solution A and solution B was obtained, which was then rested to stand for 5 minutes to 15 minutes. The mixture of the above-mentioned solution A and solution B was gently swirled and dropwise added into petri dishes containing Lenti-X-293T cells, with 2.5 ml per dish. The obtained petri dish was gently shaken back and forth to evenly distribute a mixture of DNA and calcium ions. Then the petri dish (without being rotated) was incubated for 16 hours to 18 hours in an incubator. A fresh medium was changed and the cultivation was continued. The supernatants containing virus were collected after 48 hours and 72 hours separately. The supernatants were centrifuged at 500 g for 10 minutes at 25° C. and then filtered by PES membrane (0.45 μm). The filtered supernatants containing lentivirus were transferred into ultra-clear virtus centrifuge tubes. The volume of the supernatants in each tube did not exceed ⅔ of the tube volume, and the supernatants were centrifuged at 25000 rpm at 4° C. for 2 hours. The centrifuge tubes were taken out carefully, and the supernatants were poured out, and then the centrifuge tubes were inverted to remove the residual liquid. Then the centrifuge tubes were added by 100 μl of fresh medium, sealed, stood at 4° C. for 2 hours, swirled gently every 20 minutes, and finally centrifuged at 500 g for one minute (25° C.) to collect the supernatant containing virus. The collected supernatant containing virus was cooled on ice and then stored at −80° C.

### Example 3. Preparation of ROBO1 CAR-NK Cells

The density of the NK-92 cells was adjusted to 2×10⁵/ml to 3×10⁵/ml. The virus obtained from Example 2 was added to the NK-92 cells according to a volume ratio (V/V) of virus: cell culture medium=1:5, while 8 μg/mL polyamine was added at the same time. 4 hours later, equivalent amount of fresh complete medium was added to adjust the cell density to 1×10⁵/ml for further cultivation. The next day, all the cells were centrifuged and added with fresh medium for further cultivation. Fresh medium was added every 1 to 2 days to maintain the cell density at 2×10⁵/m1 to 3×10⁵/ml. CAR antibody staining was performed after 72 hours, and ROBO1 CAR NK-92 positive cells were sorted by flow cytometry for culture expansion. The color change of the medium, cell density and cell morphology were observed daily and recorded accordingly.

After sorting the cells by flow cytometry, positive ROBO1 CAR NK-92 cells were continuously cultured in a GMP workshop, and then expanded to the required measurement for clinical use. After that, the cells were centrifugated and washed thrice (by PBS solution), then the obtained ROBO1 CAR-NK 92 cells were resuspended in normal saline for clinical treatment.

Before clinical treatment, the quality of ROBO1 CAR-NK 92 cells was tested with reference to the test methods of Pharmacopoeia to guarantee the safety of the cells. The test results are shown in Table 1.

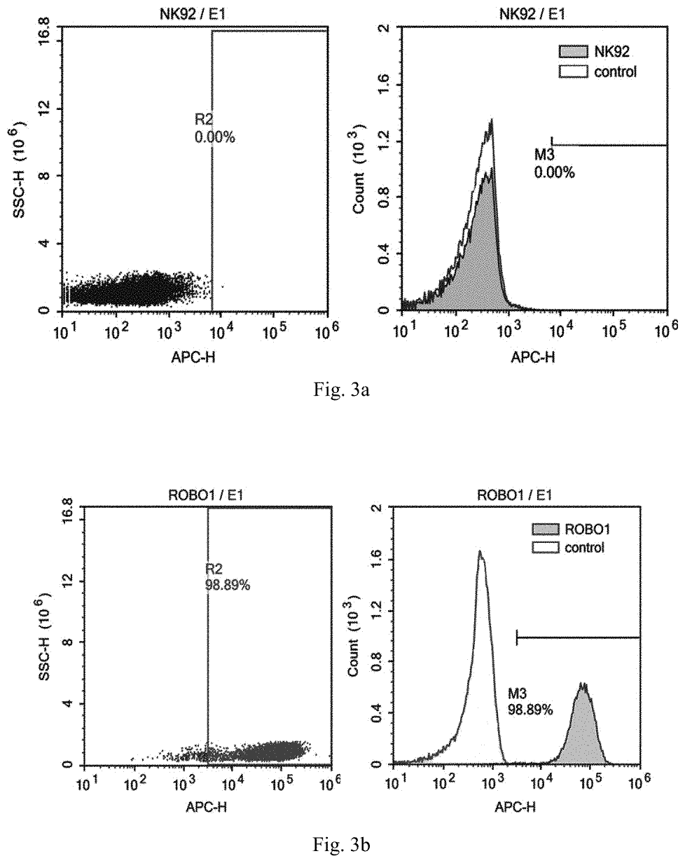

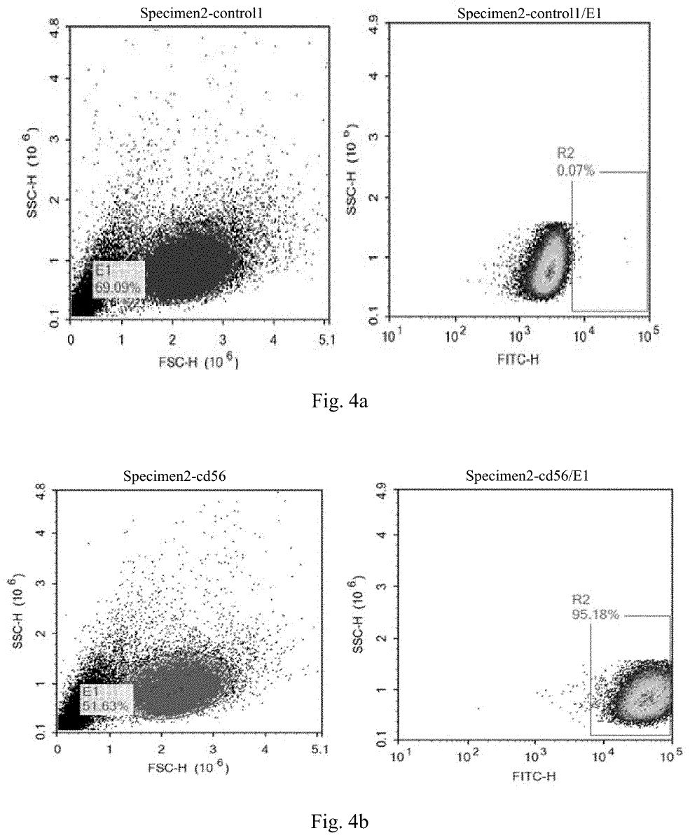

TABLE 1
Quality test of ROBO1 CAR-NK 92 cells

| Release parameters | GMP release criteria | Actual test results |
|---|---|---|
| Sterility test (liquid cultivation) | Negative | Negative |
| Sterility test (gram staining) | Negative | Negative |
| Cell viability (trypan blue staining) | >95% | 98% |
| Endotoxin (limulus reagent) | <5 EU/kg/h | <5 EU/kg/h |
| CAR positive rate (flow cytometry) | >90% | 96.31% (shown in |
| | | FIGS. 3a-b) |
| Mycoplasma DNA (PCR method) | Negative | Negative |
| CD56+ (flow cytometry) | Positive | Positive (shown in |
| | | FIGS. 4a-b) |

The positive rate of the CAR NK-92 cells was tested by flow cytometry, and the flow cytometry results were shown in FIG. 3a and FIG. 3b. In FIG. 3a and FIG. 3b, the antibodies used were shown as APC fluorescence label and represented on the abscissa. If the NK-92 cells successfully expressed CAR molecules, the signal value would increased significantly. It could be seen from FIG. 3a and FIG. 3b that the signal value of the APC fluorescence label increased significantly, indicating that the CAR molecules were successfully expressed by the NK-92 cells. The positive rate of CAR-NK92 was 98.89%.

FIGS. 4a and 4b showed results of positive rates of CD56 molecules of ROBO1 CAR-NK by flow cytometry. FIG. 4a was the control group, and FIG. 4b was the experimental group. It could be seen from FIGS. 4a and 4b that the CD56 molecule was positive, indicating that the prepared CAR-NK92 cells did not lose the CD56 molecules, and there were no other forms of differentiation, which means the basic characteristics of the NK cells had been preserved.

ROBOIM CAR-NK cells were prepared in the same way.

### Example 4. Preparation of ROBO1-iCaspase9 CAR-NK Cells

Suicide gene iCaspase9 [SEQ ID NO: 6:
ATGGGAGTGCAGGTGGAAACCATCTCCCCAGGAGACGGGCGCACCTTCCCCAAGCG
CGGCCAGACCTGCGTGGTGCACTACACCGGGATGCTTGAAGATGGAAAGAAAGTTGA
TTCCTCCCGGGACAGAAACAAGCCCTTTAAGTTTATGCTAGGCAAGCAGGAGGTGAT
CCGAGGCTGGGAAGAAGGGGTTGCCCAGATGAGTGTGGGTCAGAGAGCCAAACTGA
CTATATCTCCAGATTATGCCTATGGTGCCACTGGGCACCCAGGCATCATCCCACCACAT
GCCACTCTCGTCTTCGATGTGGAGCTTCTAAAACTGGAA (FKBP12-F36V: SEQ ID NO:
1) TCCGGAGGAGGATCCGGAGTCGAC (Linker: SEQ ID NO: 3)
GGATTTGGTGATGTCGGTGCTCTTGAGAGTTTGAGGGGAAATGCAGATTTGGCTTACA
TCCTGAGCATGGAGCCCTGTGGCCACTGCCTCATTATCAACAATGTGAACTTCTGCCG
TGAGTCCGGGCTCCGCACCCGCACTGGCTCCAACATCGACTGTGAGAAGTTGCGGCG
TCGCTTCTCCTCGCTGCATTTCATGGTGGAGGTGAAGGGCGACCTGACTGCCAAGAA
AATGGTGCTGGCTTTGCTGGAGCTGGCGCAGCAGGACCACGGTGCTCTGGACTGCTG
CGTGGTGGTCATTCTCTCTCACGGCTGTCAGGCCAGCCACCTGCAGTTCCCAGGGGC
TGTCTACGGCACAGATGGATGCCCTGTGTCGGTCGAGAAGATTGTGAACATCTTCAAT
GGGACCAGCTGCCCCAGCCTGGGAGGGAAGCCCAAGCTCTTTTTCATCCAGGCCTGT
GGTGGGGAGCAGAAAGACCATGGGTTTGAGGTGGCCTCCACTTCCCCTGAAGACGA
GTCCCCTGGCAGTAACCCCGAGCCAGATGCCACCCCGTTCCAGGAAGGTTTGAGGAC
CTTCGACCAGCTGGACGCCATATCTAGTTTGCCCACACCCAGTGACATCTTTGTGTCC
TACTCTACTTTCCCAGGTTTTGTTTCCTGGAGGGACCCCAAGAGTGGCTCCTGGTACG
TTGAGACCCTGGACGACATCTTTGAGCAGTGGGCTCACTCTGAAGACCTGCAGTCCC
TCCTGCTTAGGGTCGCTAATGCTGTTTCGGTGAAAGGGATTTATAAACAGATGCCTGG
TTGCTTTAATTTCCTCCGGAAAAAACTTTTCTTTAAAACATCA (ACasepase9: SEQ ID
NO: 2)
GAATTCGGCAGTGGAGAGGGCAGAGGAAGTCTGCTAACATGCGGTGACGTCGAGGA
GAATCCTGGCCCA (T2A: SEQ ID NO: 5)
ATGCCACCTCCTCGCCTCCTCTTCTTCCTCCTCTTCCTCACCCCCATGGAAGTCAGGC
CCGAGGAACCTCTAGTGGTGAAGGTGGAAGAGGGAGATAACGCTGTGCTGCAGTGC
CTCAAGGGGACCTCAGATGGCCCCACTCAGCAGCTGACCTGGTCTCGGGAGTCCCCG
CTTAAACCCTTCTTAAAACTCAGCCTGGGGCTGCCAGGCCTGGGAATCCACATGAGG
CCCCTGGCCATCTGGCTTTTCATCTTCAACGTCTCTCAACAGATGGGGGGCTTCTACC
TGTGCCAGCCGGGGCCCCCCTCTGAGAAGGCCTGGCAGCCTGGCTGGACAGTCAAT
GTGGAGGGCAGCGGGGAGCTGTTCCGGTGGAATGTTTCGGACCTAGGTGGCCTGGG
CTGTGGCCTGAAGAACAGGTCCTCAGAGGGCCCCAGCTCCCCTTCCGGGAAGCTCAT
GAGCCCCAAGCTGTATGTGTGGGCCAAAGACCGCCCTGAGATCTGGGAGGGAGAGC
CTCCGTGTCTCCCACCGAGGGACAGCCTGAACCAGAGCCTCAGCCAGGACCTCACCA
TGGCCCCTGGCTCCACACTCTGGCTGTCCTGTGGGGTACCCCCTGACTCTGTGTCCAG
GGGCCCCCTCTCCTGGACCCATGTGCACCCCAAGGGGCCTAAGTCATTGCTGAGCCT
AGAGCTGAAGGACGATCGCCCGGCCAGAGATATGTGGGTAATGGAGACGGGTCTGTT
GTTGCCCCGGGCCACAGCTCAAGACGCTGGAAAGTATTATTGTCACCGTGGCAACCT
GACCATGTCATTCCACCTGGAGATCACTGCTCGGCCAGTACTATGGCACTGGCTGCTG
AGGACTGGTGGCTGGAAGGTCTCAGCTGTGACTTTGGCTTATCTGATCTTCTGCCTGT
GTTCCCTTGTGGGCATTCTTCATCTTCAAAGAGCCCTGGTCCTGAGGAGGAAAAGAA
AGCGAATGACTGACCCCACCAGGAGATTCTTCAAAGTGACGCCTCCCCCAGGAAGC
GGGTGA (CD19: SEQ ID NO: 4)]

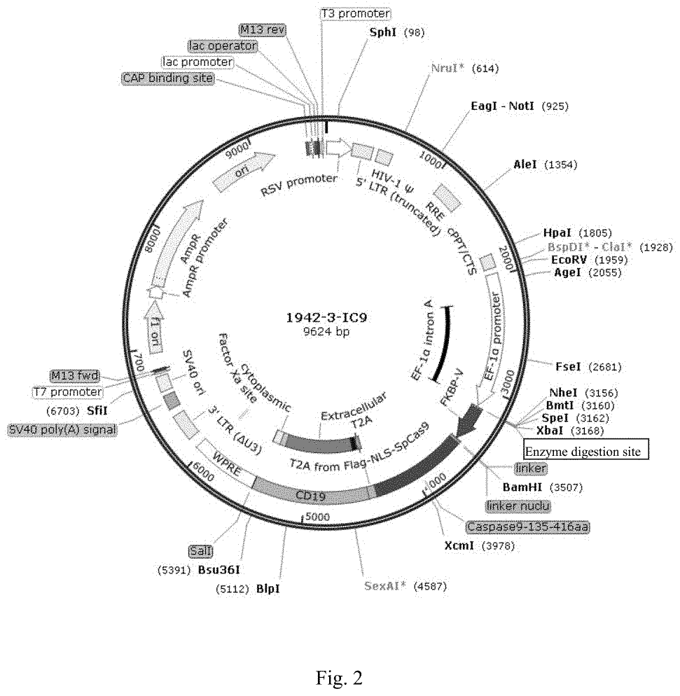

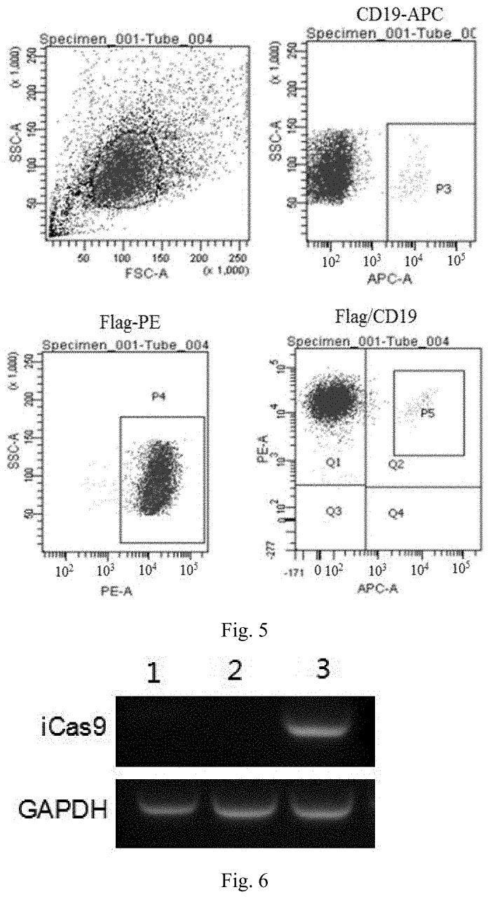

was synthesized, and then cloned into a lentiviral vector to obtain a lentiviral plasmid vector (1942-3-IC9) (shown in FIG. 2, with a nucleotide sequence shown in SEQ ID NO: 7) carrying the suicide gene iCaspase9, and then the virus was packaged by a three-plasmid system (aided by plasmids pMD2.G and pCMVR8.74) (a nucleotide sequence of the packaged virus was shown in SEQ ID NO: 7). Then, the ROBO1 CAR-NK cells (or ROBO1M CAR-NK cells, wherein the ROBO1M CAR-NK cells were used in the following examples) prepared in Example 3 were infected by means of virus infestation, and then the ROBO1 and iCaspase9 double-positive cells, i.e., the ROBO1 CAR-NK cells (ROBO1-iCaspase9 CAR-NK cells) carrying the suicide gene were sorted by flow cytometry. The screened cells were cultured, and then the positive rate of the ROBO1 CAR-NK cells carrying the suicide gene was tested from the cultured cells by flow cytometry. The results were shown in FIG. 5. It could be seen from the figure that the signal values of APC and PE fluorescence label were significantly increased, and the ROBO1 CAR-NK cells carrying the suicide genes (i.e., ROBO1-iCaspase9 CAR-NK cells) were successfully obtained.

In addition, the RNA of the cultured ROBO1-iCaspase9CAR-NK cells was extracted, and a mRNA level of the suicide gene fragment was tested by RT-PCR. Meanwhile, the NK-92 cells and the ROBO1 CAR-NK cell were used as controls, and the results were shown in FIG. 6, which showed that the ROBO1 CAR-NK cells (ROBO1-iCaspase9CAR-NK cells) carrying the suicide gene may be verified at the mRNA level.

### Example 5. Sequencing of ROBO1-iCaspase9 CAR-NK Cells

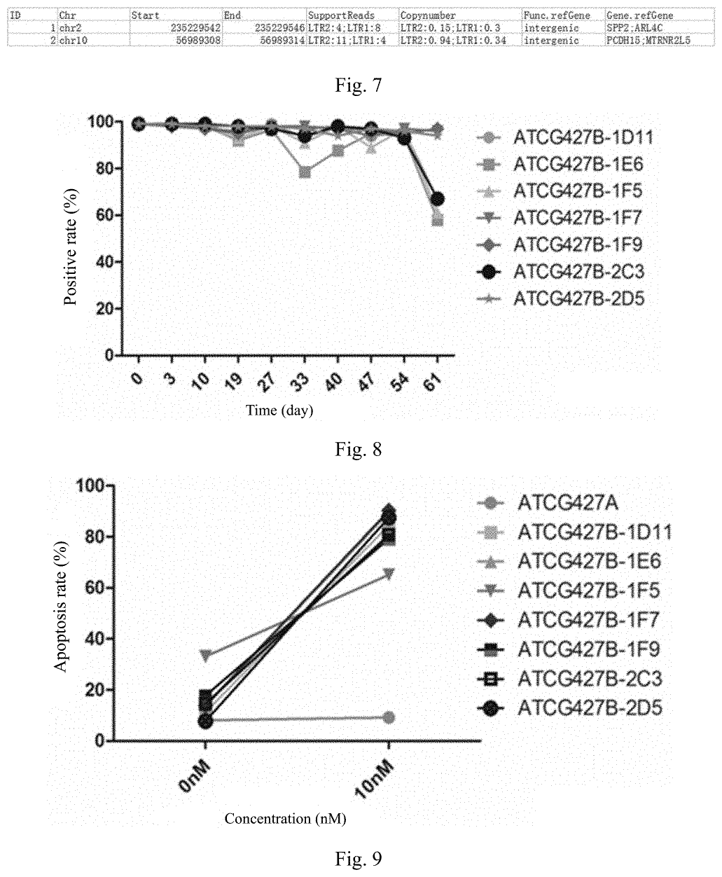

Whole genome sequencing was carried out on the ROBO1-iCaspase9CAR-NK cells prepared in Example 4, and the insertion sites of the ROBO1-iCaspase9CAR-NK cells in the genome of the NK-92 cell were tested. The sequencing results were shown in FIG. 7. After PCR verification, the sequencing results showed that No. 2 chromosome was inserted into the gene iCaspase9 and No. 10 chromosome was inserted into the gene ROBO1.

### Example 6. Stability Screening of ROBO1-iCaspase9 CAR-NK Cells

The ROBO1-iCaspase9CAR-NK cells constructed in Example 4 were cultured. The ROBO1-iCaspase9CAR-NK cells of different clones were respectively named ATCG427B-1D11, ATCG427B-1E1, ATCG427B-1F5, ATCG427B-1F7, ATCG427B-1F9, ATCG427B-2C3 and ATCG427B-2D5. Then, the positive rates of flag antibody Flag and CD19 in these cells were tested by flow cytometry (specifically, the cells were stained with flow antibodies Flag and CD19 in dark for 15 minutes, then washed twice with PBS, and tested on the machine), which were used as a cell stability index. The results were shown in FIG. 8.

Different amounts of medicament AP1903 were respectively added into the ROBO1-iCaspase9CAR-NK cells prepared in Example 4 (the final concentration of the medicament AP1903 was 0 nM and 10 nM) for 4 hours to test the effect thereof on apoptosis of the ROBO1-iCaspase9CAR-NK cells. The results were shown in FIG. 9.

It could be seen from the above FIGS. 8-9 that ATCG427B-1F7 and 2D5 were two subclones which are stable and safe in vitro.

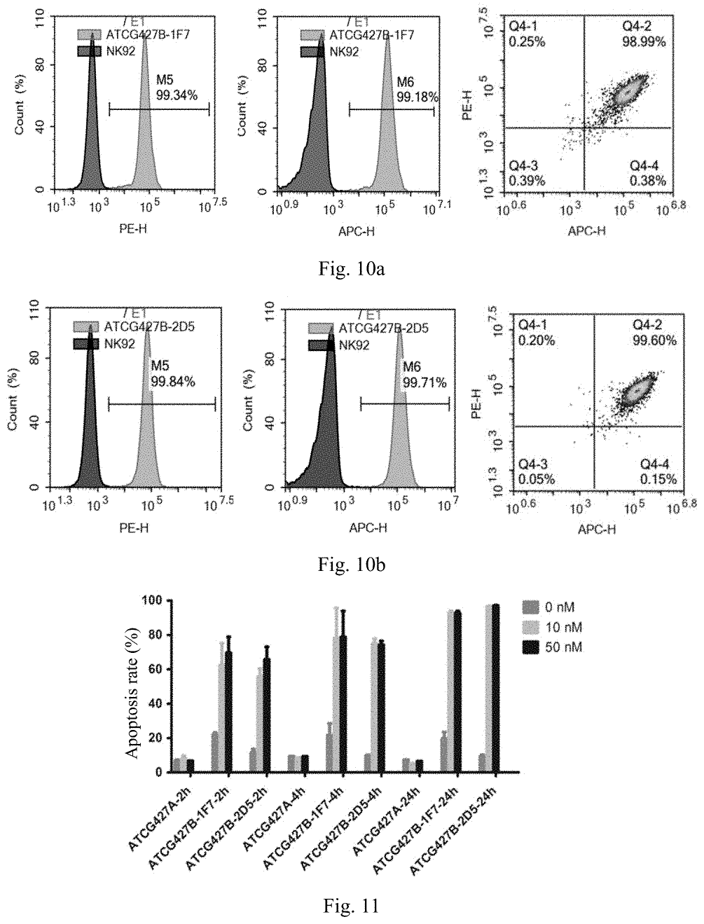

Then, two best subclones of the ROBO1 CAR-NK cells carrying the suicide gene after passing the positive rate by flow cytometry were selected for subsequent experiment and continuous culture, and the two subclones cells were sequenced and verified at the same time, and the sequencing results were correct. Finally, the positive rates of ROBO1 and isapase9 of the verified subclones were tested respectively by flow cytometry, and the results were shown in FIGS. 10a-10b. It could be seen from the figures that ATCG427B-1F7 and 2D5 were two stable subclones screened.

### Example 7. Detection of Apoptosis of ROBO1-iCaspase9CAR-NK Cells Induced by Medicament AP1903

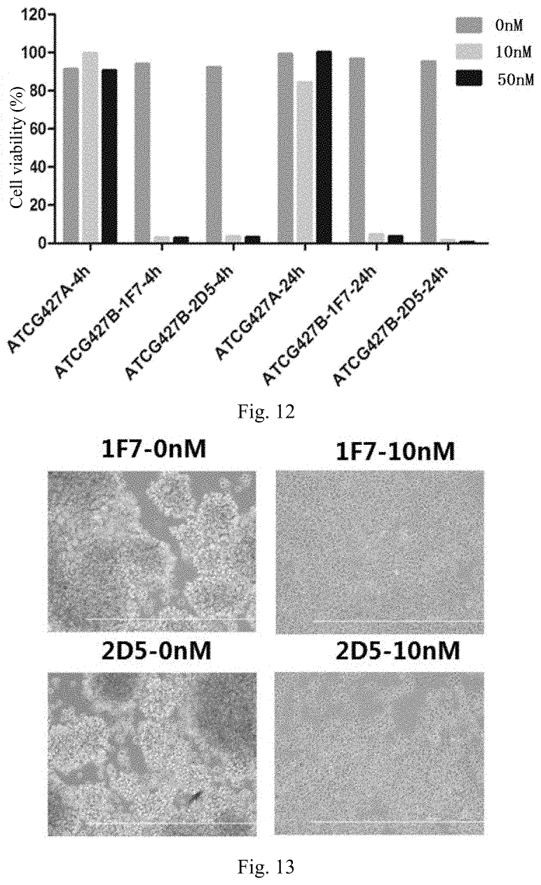

Different amounts of medicament AP1903 were respectively added into the ROBO1 CAR-NK cells carrying the suicide gene (ATCG427B-1F7 and 2D5) after screening in Example 6 to test the effect thereof on apoptosis of the ROBO1 CAR-NK cells carrying the suicide gene (denoted with ATCG427B). Meanwhile, the medicament AP1903 was added into the ROBO1 CAR-NK cells as control, and divided into three experimental groups, i.e., cells treated by the medicament AP1903 with different concentrations (0 nM, 10 nM and 50 nM) for 2 hours, 4 hours and 24 hours. Then, apoptosis was detected, and the results were shown in FIG. 11. The experimental groups respectively treated for 4 hours and 24 hours were continuously cultured with fresh medium, and the cell viability was counted three days later, and the results were shown in FIG. 12. In addition, the 0 nM and 10 nM experimental groups treated for 4 hours were cultured in fresh medium for 24 hours and then photographed, and the results were shown in FIG. 13.

It could be seen from FIG. 11 that the apoptosis rate of the ROBO1-iCaspase9 cells could reach 60% when the cells were treated with the medicament AP1903 for 2 hours at the concentration of 10 nM in vitro, reach 80% when being treated for 4 hours, and more than 95% of the cells were apoptotic when being treated for 24 hours, while the ROBO1CAR-NK cells were not apoptotic basically.

It could be seen from FIG. 12 that, after being treated with the medicament AP1903, the survival rate of the ROBO1CAR-NK cells was over 80%, while all the ROBO1-iCaspase9 CAR-NK cells of the present invention were apoptotic basically.

It could be seen from FIG. 13 that, after being treated with the medicament AP1903 at 10 nM, the cells were basically apoptotic through observation after 24 hours.

The above results indicate that the medicament AP1903 can effectively kill the ROBO1-iCaspase9CAR-NK cells within 24 hours, but has no effect on the ROBO1-iCaspase9CAR-NK cells, indicating that applying the ROBO1-iCaspase9CAR-NK for treating cancer can induce cell apoptosis under the induction of the medicament AP1903, thus better controlling the CAR-NK cells and further increasing the clinical safety.

### Example 8. Detection of ROBO1 Expression in Different Breast Cancer Cell Strains

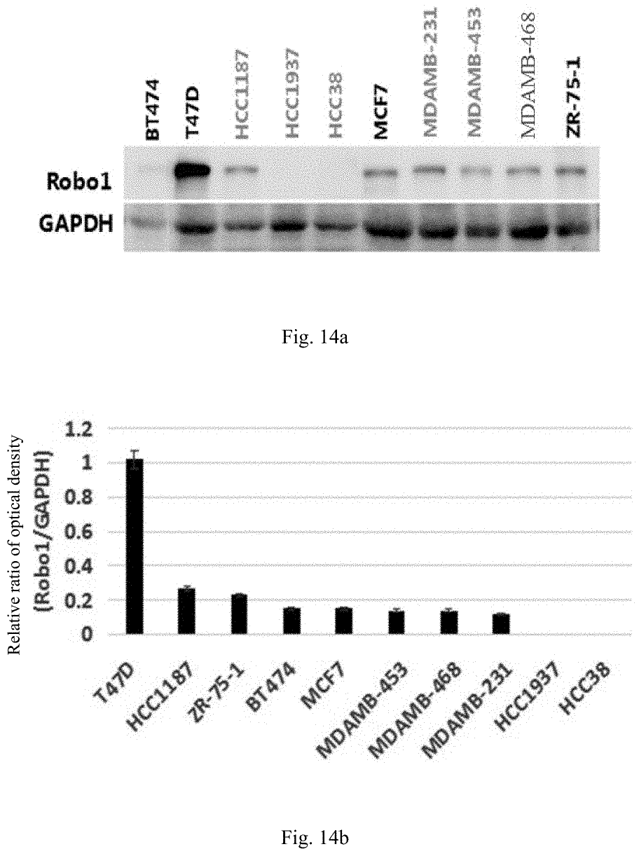

After different breast cancer cells BT474, T47D, HCC1187, HH1937, HCC38, MCF7, MDAMB-231, MDAMB-453, MDAMB-468 and ZR-75-1 were cultured, the expression of ROBO1 in different breast cancer cells was tested by Western Blot, and the results were shown in FIG. 14a. Moreover, a ratio of an optical density of ROBO1 determined by Western Blot to that of control GAPDH was analyzed by ImageJ software, and the results were shown in FIG. 14b.

The results showed that the expression of ROBO1 was different in different breast cancer cell strains, and the highest expression level was found in T47D.

### Example 9. Killing Test of ROBO1-iCaspase9 CAR-NK Cells

1. ROBO1 CAR-NK Cells Carrying a Suicide Gene for Killing Breast Cancer Cells

A CFSE staining method (referring to: A Mathematical Model of Natural Killer Cell Activity, Anna Scherbakova, 1, 2 Helen Lust, 1, 2 Hele Everaus, 1, 2, 3 Alar Aints1, 2, 4*) was used to test the killing effect of the ROBO1 CAR-NK cells carrying the suicide gene (ATCG427B-1F7, ATCG427B-2D5) screened in Example 5, and the ROBO1 CAR-NK and NK-92 cells were used as controls at the same time. Different breast cancer cells included BT474, T47D, HCC1187, HCC1937, MCF7, MDAMB-231, MDAMB-453, MDAMB-468 and ZR-75-1.

The experimental operation method was as follows:

(1) Target cells were treated in advance, and 1 μl of CFSE was added for 1×10⁶ cells, then the cells were incubated in a dark incubator for 15 minutes, and stopped with 10% BSA, washed with PBS twice, and then 500 μl of tumor cell suspension (8×10⁴ cells/well) was added into a 24-well plate. The culture plate was pre-cultured in an incubator for 12 hours.

(2) 500 μl of effector cells were added according to a ratio of the effector cells to the target cells of 0.5:1 and 1:1, three complex wells were placed in each experiment, and the effector cells and the target cells were co-incubated for 2 hours.

(3) After 2 hours, the supernatant was transferred to a 1.5 EP tube, and 200 μl of pancreatin was added to each hole. After digestion for 1 minute, the cells were beaten with the corresponding transferred supernatant, centrifuged, resuspended in PBS, added with 1 μl of 7-AAD, incubated in dark for 15 minutes, and tested on a machine.

(4) Killing rate=(killing rate of target cells−spontaneous mortality)/(1−spontaneous mortality)×100%.

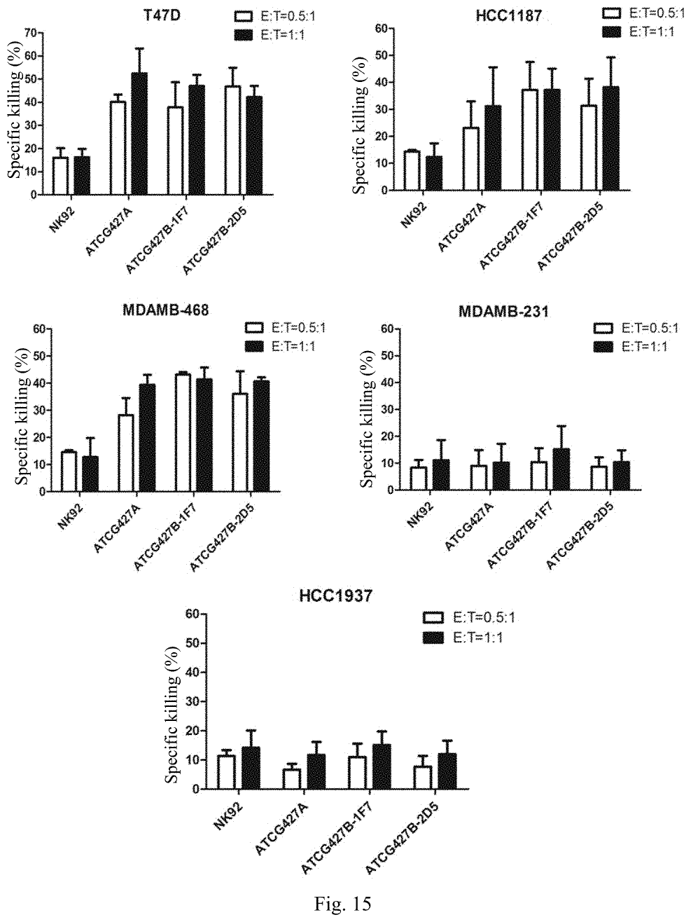

The results as shown in FIG. 15, It could be seen from the figure that different cancer cells have different killing effects in vitro, and cells with high ROBO1 expression, such as T47D cells, have better killing effects; However, cells that do not express ROBO1, such as HCC1937, have poor killing effect; while the killing effect of MDAMB-231 cells was relatively poor even through the ROBO1 was expressed therein, and the reasons might be as follows: firstly, the ROBO1 proteins expressed by the MDAMB-231 was relatively low; secondly, PDL1 molecules were highly expressed in the MDAMB-231 cells, and a PD1-PD-L1 signaling pathway would inhibit the killing of the effector cells in the killing process; and thirdly, MICA/B was basically not expressed in MDAMB-231 itself, while the NK cells partly depended on the combination of activated receptors such as NKG2D and ligand MICA/B to play a killing role, so the non-expression of MICA/B would also weaken the killing of the MDAMB-231 cells. In addition, there might be other immune mechanisms regulating the killing of MDAMB-231 by the effector cells. In a word, the mechanism of the ROBO1-iCaspase9CAR-NK cells killing and controlling the MDAMB-231 cells needs further verification.

Therefore, the above experimental results showed that the specific killing effect of the ROBO1 CAR-NK cells carrying the suicide gene of the present invention was positively correlated with the expression level of ROBO1.

2. ROBO1 CAR-NK Cells Carrying a Suicide Gene for Killing Different Cancer Cells

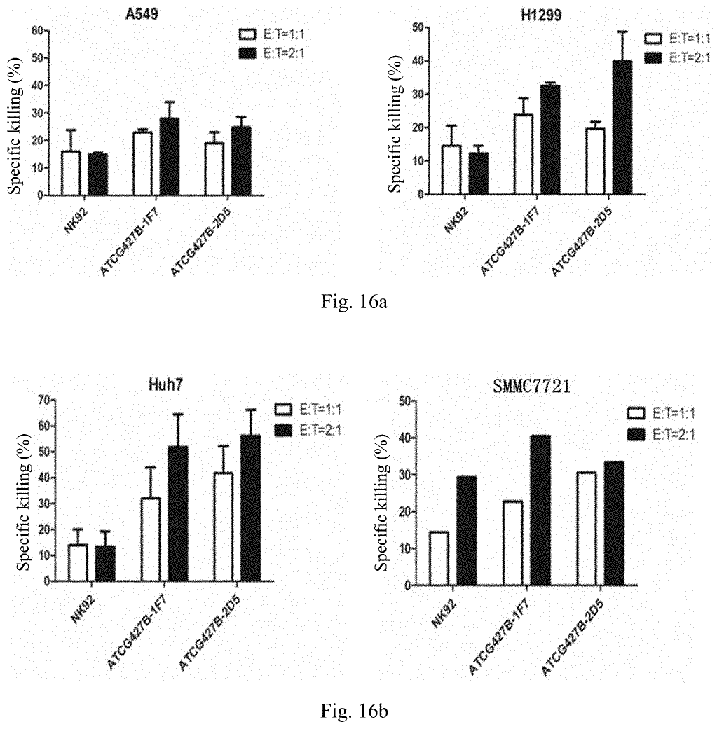

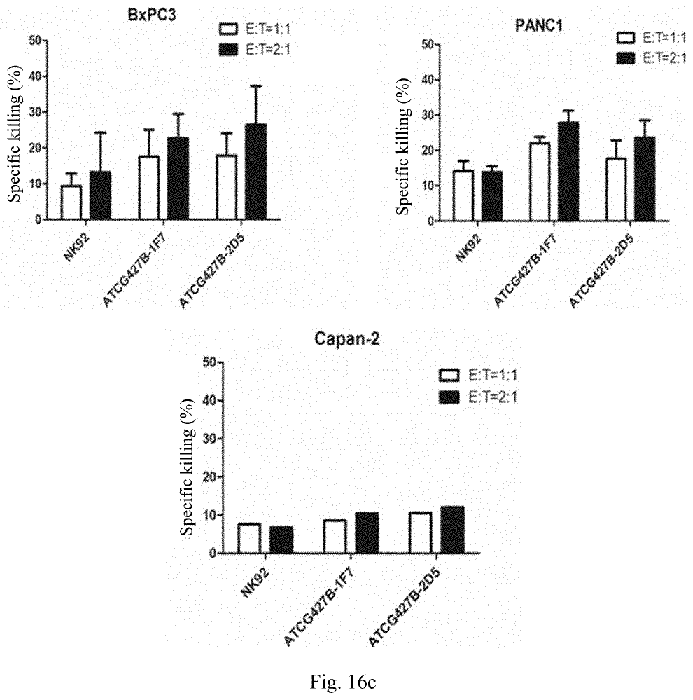

Other types of tumor cells were killed by the same method as in the above 1 for 2 hours, and the killing effects of the ROBO1 CAR-NK cells (ATCG427B-1F7, ATCG427B-2D5) carrying the suicide gene screened in Example 5 on different tumor cells were tested. Different tumour cells included lung cancer cells A549 and H1299; hepatoma cells Huh7 and SMMC7721; and pancreatic cancer cells BxPC3, PANC1 and Capan-2, which were cultured and then killed. The results were shown in FIG. 16a-c, from which it could be seen that the ROBO1-iCaspase9 cells had better killing effects on the lung cancer and the liver cancer.

3. Killing of the ROBO1 CAR-NK Cells Carrying the Suicide Gene on Normal Breast Cells and PBMC

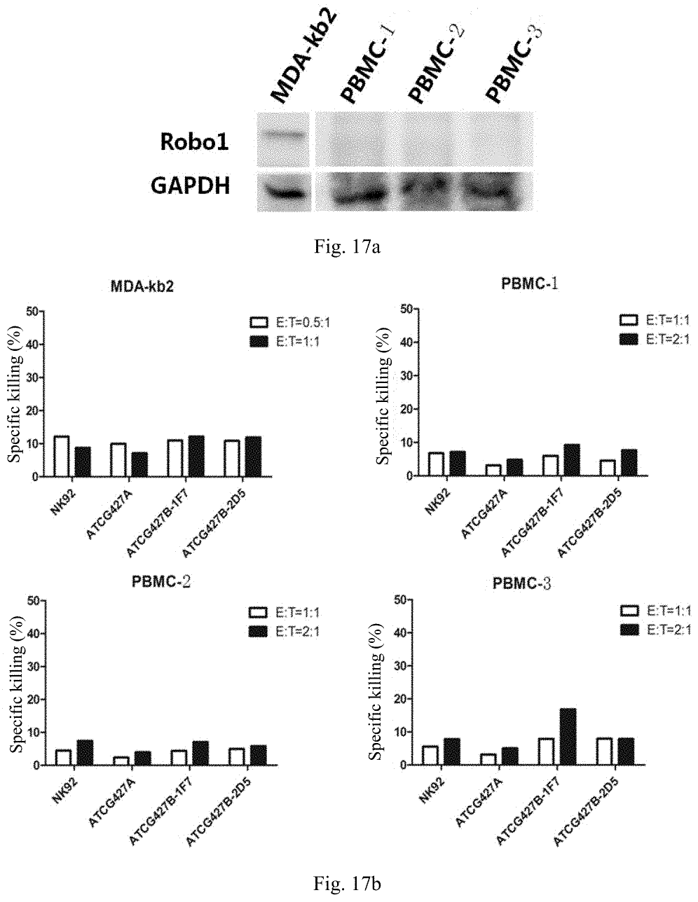

Three normal people were selected randomly, and 50 ml of fresh blood was taken from each, then the blood was diluted with PBS at a ratio of 1:1; a new centrifuge tube was taken and added with Ficoll (lymphocyte separation solution), and tilted at 45 degrees. The diluted blood was slowly added above the liquid level of Ficoll according to a ratio of Ficoll to blood of 2:1, and centrifuged at 2000 rpm. After 30 minutes, a pipette was directly inserted into a mist layer to suck out the mist layer (i.e., mononuclear cell layer) gently, put into a new centrifuge tube, and washed; then the supernatant was discarded, and 1 ml of RPMI-1640 culture medium was added, blew and blended to prepare PBMC cell suspensions which were respectively denoted with PBMC-1, PBMC-2 and PBMC-3. The ROBO1 expression in normal breast cells and PBMC was tested. The results were shown in FIG. 17a. In addition, the killing potency of the ROBO1-iCaspase9 cells on PBMC-1, PBMC-2, PBMC-3 and MDA-kb2 normal breast cells was tested by the same method as above 1, and the killing rates of NK92 and ROBO1 CAR-NK on PBMC-1, PBMC-2, PBMC-3 and MDA-kb2 cells were compared at the same time in the killing process. The results were shown in FIG. 17b. It could be seen from the figure that the ROBO1-iCaspase9CAR-NK cells have no significant killing effect on the normal breast cells and PBMC in vitro. Thus, the safety of the ROBO1 CAR-NK cell medicament carrying the suicide gene was further proved.

4. Killing of PBMC on ROBO1 CAR-NK Cells Carrying the Suicide Gene

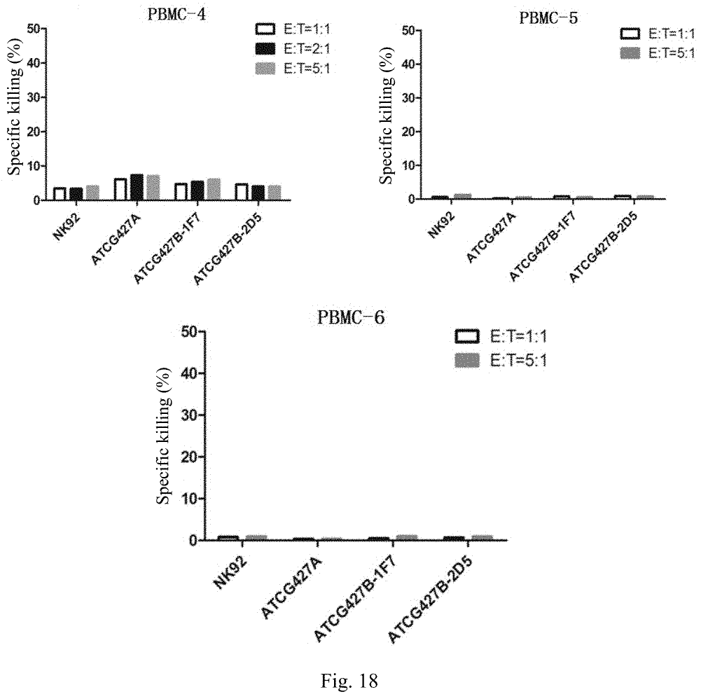

Considering that the ROBO1 CAR-NK cells were infused into human body as allogeneic cells, PBMC immune cells in vivo might kill the allogeneic cells. To verify whether the ROBO1 CAR-NK cells would be cleared by PBMC, in vitro, three normal people were randomly selected to collect blood, and PBMC cells were obtained after separation (the preparation method was the same as above), which were denoted with PBMC-4, PBMC-5 and PBMC-6 respectively. In addition, the killing of PBMC-4, PBMC-5 and PBMC-6 on the ROBO1-iCaspase9CAR-NK cells was tested in the same way as in the above 1, and the killing of PBMC on the NK92 and ROBO1 CAR-NK cells was compared in each group of experiments. The results were shown in FIG. 18. It could be seen from FIG. 18 that PBMC had no significant killing effect on the ROBO1-iCaspase9 cells in vitro. Therefore, it was further proved that the ROBO1 CAR-NK cell medicament carrying the suicide gene could play a better role in specific killing in vivo.

The above experimental results showed that the killing effect of the ROBO1-iCaspase9 CAR-NK cells of the present invention was equivalent to that of the ROBO1 CAR-NK cells, which could effectively kill tumor cells, and had less cytotoxicity and better killing effect than the CAR-T cells. Moreover, due to the addition of the suicide gene (iCaspase9 switch element), the safety could be further improved. In addition, experiments showed that this cells did not kill normal cells, and the normal cells do not kill this cells, which further illustrated the safety of the ROBO1-iCaspase9 CAR-NK cells.

### Example 10. Preparation of ROBO1-EGFRt-CAR-NK Cells and Screening of Monoclonal Cell Strains

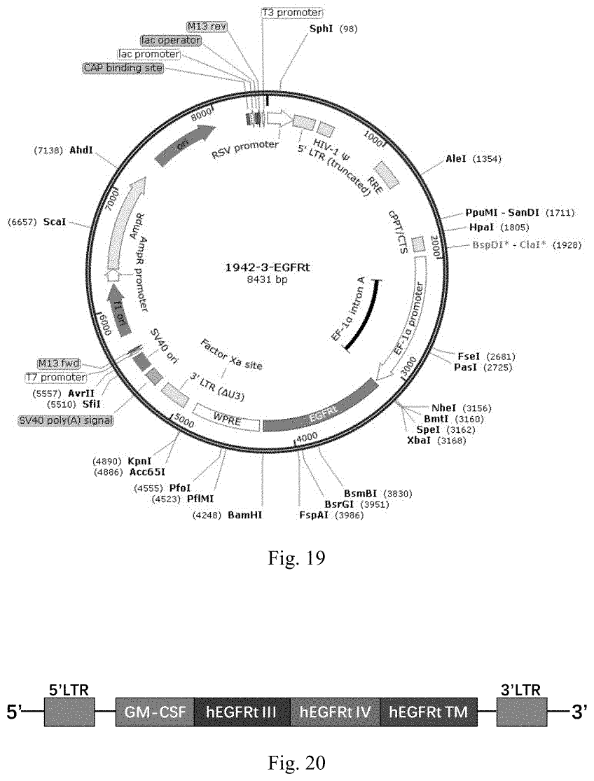

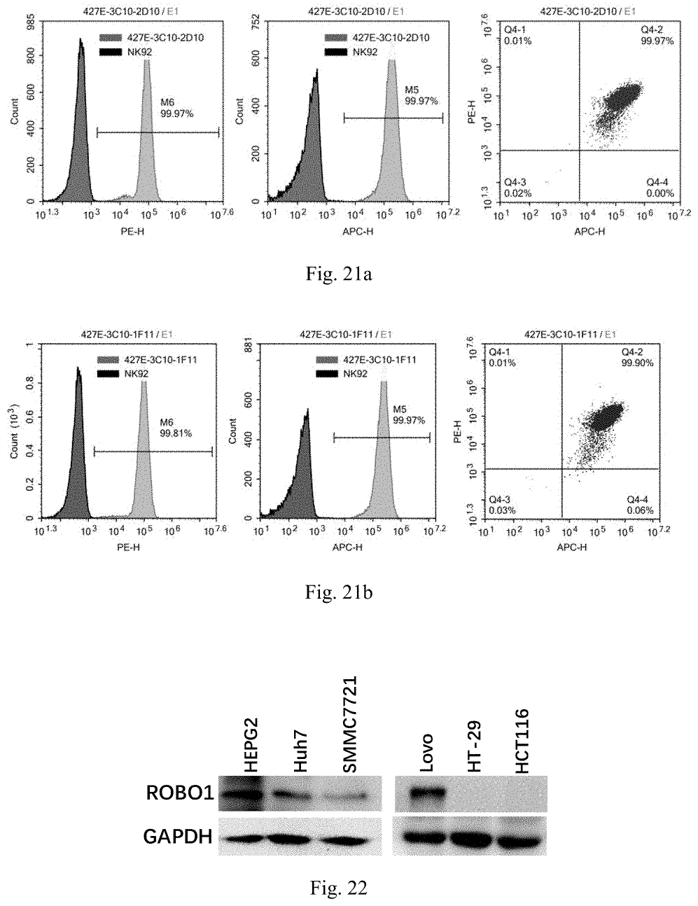

Suicide gene EGFRt
(SEQ ID NO: 12
TCTAGAATGTTGCTGCTTGTAACTTCTCTCCTTCTTTGCGAGTTGCCCC
ATCCTGCGTTCCTCCTTATTCCCAGGAAGGTATGCAATGGGATCGGTAT
AGGAGAGTTCAAGGATTCCCTTTCTATCAACGCTACGAATATAAAGCAC
TTCAAGAACTGTACGTCCATCAGTGGAGACCTGCATATATTGCCGGTGG
CGTTCCGAGGGGACAGTTTTACCCACACGCCCCCTCTCGACCCACAGGA
GCTGGATATCTTGAAGACCGTGAAGGAGATAACTGGCTTTCTTCTCATT
CAGGCGTGGCCGGAAAATAGGACAGACTTGCACGCCTTTGAAAACTTGG
AAATTATACGAGGGCGGACAAAACAACACGGTCAATTCAGCCTGGCCGT
TGTATCCCTCAATATCACTAGCTTGGGTCTCCGAAGTCTGAAAGAAATA
AGTGACGGGGACGTTATAATTTCTGGGAACAAGAACCTCTGCTACGCAA
ACACAATAAACTGGAAAAAATTGTTTGGAACTAGCGGGCAGAAAACTAA
GATCATTAGTAACAGAGGCGAGAATAGTTGCAAAGCCACCGGACAAGTG
TGCCATGCACTTTGCAGCCCCGAGGGTTGTTGGGGCCCTGAACCACGGG
ATTGCGTGTCATGCAGAAACGTCTCACGAGGTCGCGAGTGTGTCGACAA
ATGTAACCTGCTTGAAGGGGAGCCTCGCGAATTCGTAGAAAACAGCGAG
TGCATTCAATGCCACCCAGAGTGTCTCCCCCAGGCCATGAACATCACCT
GTACAGGACGGGGGCCAGATAACTGTATTCAATGCGCACACTATATAGA
TGGACCACATTGTGTGAAAACATGTCCCGCAGGGGTCATGGGTGAGAAC
AACACGCTCGTTTGGAAATATGCAGATGCCGGGCATGTATGCCACCTCT
GTCACCCGAACTGCACTTATGGGTGCACTGGGCCCGGCCTGGAAGGATG
CCCCACCAACGGACCCAAGATTCCCTCCATAGCGACCGGAATGGTTGGA
GCCTTGCTTCTTCTTCTGGTAGTGGCGCTCGGGATCGGGTTGTTCATGT
AAGGATCC,

wherein the underlined parts in italics at the beginning and the end were restriction sites) (with a simplified structure shown in FIG. 20) was synthesized, and then cloned into a lentiviral vector to obtain a lentiviral vector (called 1942-3-hEGFRt) shown in FIG. 19 (with a nucleotide sequence shown in SEQ ID NO: 13), and then the virus was packaged by a three-plasmid system (aided by plasmids pMD2.G and pCMVR8.74). Then, the ROBO1 CAR-NK cells (or ROBO1M CAR-NK cells, wherein the ROBO1M CAR-NK cells were used in the following examples) prepared in Example 3 were infected by means of virus infestation, and then the ROBO1 and EGFRt double-positive cells, i.e., the ROBO1 CAR-NK cells (ROBO1-EGFRt CAR-NK cells, also called 427E) carrying the suicide gene were sorted by flow cytometry. The screened cells were cultured in a monoclonal screening manner, and then the positive rate of the ROBO1 CAR-NK cells carrying the suicide gene was tested from the cultured cells by flow cytometry. The results were shown in FIGS. 21a-b. It could be seen from the figures that the signal values of APC and PE fluorescence label were significantly increased, and two ROBO1 CAR-NK monoclonal cells (i.e., ROBO1-EGFRt CAR-NK cells, i.e, 427E) carrying the suicide genes were successfully obtained by screening, which were respectively named ROBO1-EGFRt-3C10-2D10 and ROBO1-EGFRt-3C10-1F11 (also called 427E-3C10-2D10 and 427E-3C10-1F11 respectively). The mother clone of the two monoclonal cells was ROBO1-EGFRt-3C10 (427E-3C10).

### Example 11. Killing Experiment of ROBO1-EGFRt-CAR-NK Cells

Tumor-specific killing experiments were carried out on the monoclonal cell strain prepared in Example 10 and the mother clone of the two monoclonal cell strains. The experimental process was as follows: firstly, the expression level of ROBO1 protein in different types of cancer cells except the breast cancer cells was tested by Western, and the specific steps were as follows: collecting different cancer cell strains, extracting the total protein for lysis, quantifying the total protein with a BCA kit, then subjecting the ROBO1 protein to electrophoresis by SDS-PAGE gel and membrane transferring; and finally, testing the ROBO1 protein by developing. The expression of ROBO1 in some breast cancer cells was referred to the test results in FIG. 14a, while the test results of colorectal cancer cells such as HT-29, LoVo and HCT116 and liver cancer cells such as SMMC7721, Huh7 and HEPG2 were shown in FIG. 22. The results showed that the expression of the ROBO1 protein in different types of cancer cells was obviously different.

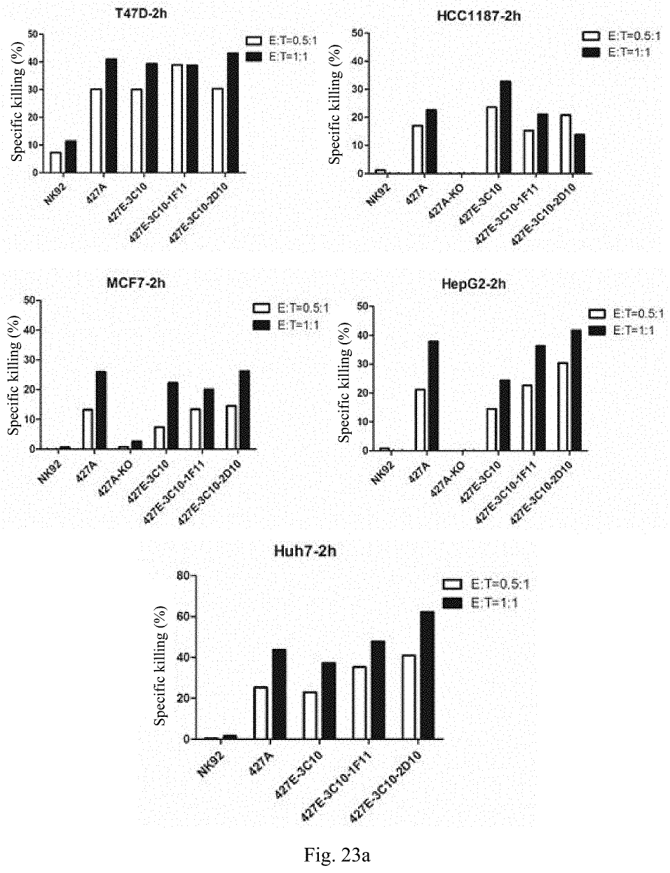

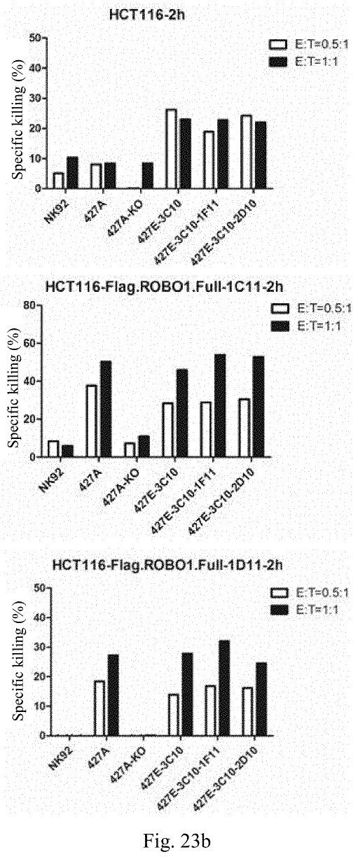

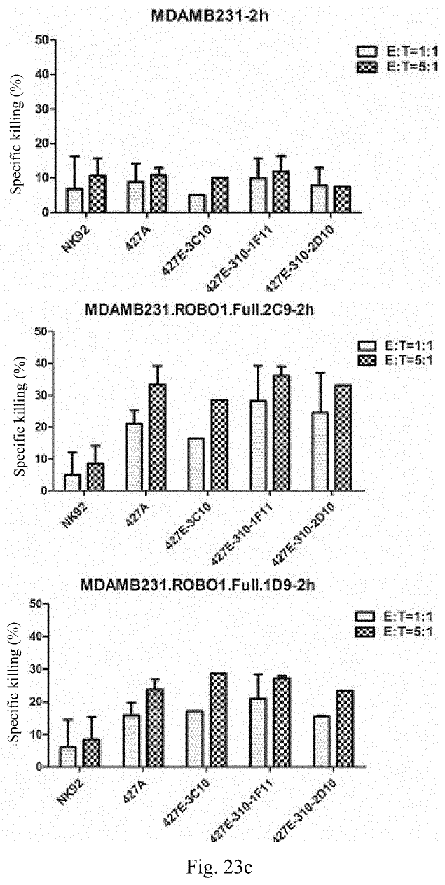

Different cancer cells including T47D, HCC1187, MCF7, HepG2 and Huh7 were taken for culture, and meanwhile, ROBO1-EGFRt-3C10, ROBO1-EGFRt-3C10-2D10 and ROBO1-EGFRt-3C10-1F11 were cultured and killed in vitro for 2 hours according to an effector-to-target ratio of 0.5:1 and 1:1, respectively, and NK92 and 427A were added as control groups, wherein a 427A-KO experimental group (427A-KO referred to “knocking out the gene sequence of a ROBO1-scFV target in CAR in the form of Crisp Cas9 gene editing, keeping other signal transduction sequences, and then knocking the CAR into a NK92 cell, that is, the NK cell in which the CAR sequence without the ROBO1 target is knocked on the same genome insertion site as CAR in 427A”) was also added for the killing experiments of HCC1187, MCF7 and HepG2. According to “Methodology study of evaluating immune cell therapeutic products cytotoxic potency in vitro” issued by National Institutes for Food and Drug Control, RTCA Systems real-time cell analysis system was a new cell testing system ACEA. The results were shown in FIG. 23a. Through killing analysis, it could be found that the ROBO1-EGFRt-CAR-NK cells showed strong killing effect, and the killing effect thereof on solid tumors was obvious, and the specific killing effect of the cells was positively correlated with the ROBO1 expression level, thus proving the effectiveness of the ROBO1-EGFRt CAR-NK cell medicament.

Meanwhile, in order to prove the specificity of ROBO1 CAR, a ROBO1-overexpressed monoclonal cell strain (HCT116-ROBO1) model was also constructed in vitro, and HCT116 was used as a control, and then the killing experiments were carried out according to the above method. The results were shown in FIG. 23b. It could be seen from the figure that in wild-type HCT116, the killing effect of 427E was slightly stronger than that of NK92, wherein the 427A-KO after knocking out ROBO1 target has no difference from NK92 and 427A. However, in the ROBO1-overexpressed HCT116 monoclonal cell strain, the killing effect of 427A-KO was obviously weakened compared with that of 427A and E; meanwhile, the killing activity of 427E in the overexpressed HCT116 cell strain was obviously enhanced compared with that of the wild type HCT116, thus effectively proving the targeting and specificity of ROBO1 in the ROBO1-EGFRt-CAR-NK cell of the invention.

In addition, a ROBO1-overexpressed monoclonal cell strain (MDAMB-231-ROBO1) model was constructed by the same method, and MDAMB-231 was used as a control group, and then the killing experiments were carried out according to the above method. The results were in FIG. 23c. Compared with the control group MDAMB-231, the killing activity and specificity ROBO1-overexpressed monoclonal cell strain (MDAMB-231-ROBO1) model were obviously improved.

### Example 12. Safety Experiment of ROBO1-EGFRt-CAR-NK Cells

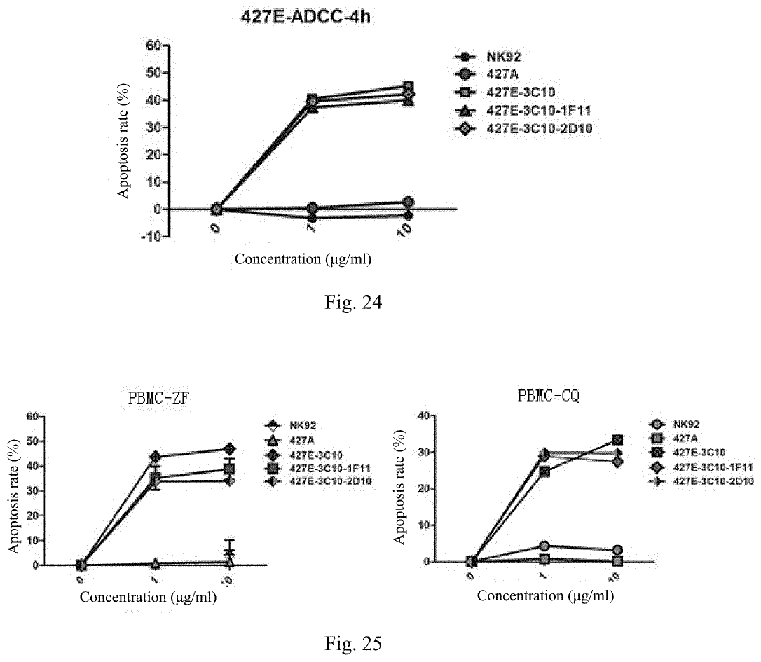

Safety study was to investigate the sensitivity of the ROBO1-EGFRt CAR-NK cells to a suicide switch inducer (cetuximab) in the presence of killer/effector cells. The effectiveness of the suicide switch was evaluated by inducing apoptosis of the ROBO1-EGFRt CAR-NK cells (the monoclonal cell strains prepared in Example 10 and the mother clone of the two monoclonal cell strains) with cetuximab, and adding NK92-CD16 (high affinity) effector cells constructed in vitro. The concentration, target ratio, action time and action system of the effector cells were investigated respectively. Finally, optimal action conditions of the inductor cetuximab were as follows: 4 hours in vitro, the action concentration was 1 μg/ml, the effector-to-target ratio of NK92-CD16 (high affinity) to ROBO1-EGFRt CAR-NK cells was 25:1 (E:T). The test method was CFSE (referring to: A Mathematical Model of Natural Killer Cell Activity, Anna Scherbakova, 1, 2 Helen Lust, 1, 2 Hele Everaus, 1, 2, 3 Alar Aints 1, 2, 4*), and the cell apoptosis potency was tested by 7-AAD/CFSE double staining. The specific method was as follows: the constructed NK92-CD16 effector cells were labeled with CFSE in advance, then the ROBO1-EGFR CAR-NK and NK92-CD16 cells were plated according to the effector-to-target ratio, and then cetuximab with the action concentrations of 0 μg/ml, 1 μg/ml and 10 μg/ml were added respectively. After 4 hours of action, the cells were stained with 7-AAD for 10 minutes, and then tested by flow cytometry (three parallel repetitions). The test results were shown in FIG. 24. It could be seen from the results that the apoptosis potency induced by cetuximab was between 40% and 50% in the selected subcloned cell strains.

In order to better simulate the mechanism of ADCC in vivo and evaluate the effectiveness of the suicide switch more accurately, in vitro, peripheral bloods from two normal people were extracted, and PBMC were separated, which were denoted with PBMC-ZF and PBMC-CQ. According to the above experimental methods and conditions, PBMC-ZF and PBMC-CQ were incubated with NK92/427A/427E cell strains, and cetuximab (with action concentrations of 0 μg/ml, 1 μg/ml and 10 μg/ml, respectively) were added at the same time, and the effector-to-target ratio was 25:1. After 4 hours of action, the killing potency (referring to FIG. 25) of 427E cell strains by PBMC was tested by flow cytometry under the action of cetuximab. It could be seen from the results that under the same effector-to-target ratio and concentration, the potency of cell killing induced by PBMC of different people were different, which was caused by the differences of immune cell compositions and functions of different people. However, compared with the 427A and NK92 cells, the potency of the cell killing induced by 427E was obviously enhanced. On the whole, when PBMC was used as effector cells (the effector-to-target ratio was 25:1), the apoptosis potency of ATCG427E-3C10-1F11 and ATCG427E-3C10-2D10 induced by cetuximab (with a action concentration of 1 μg/ml) ranged from 30% to 40%, which was slightly worse than that induced by NK92-CD16 serving as effector cells, and this was consistent with the data reported in existing literatures and patents, and even, our effect was more obvious.

In conclusion, by the evaluation on the effect of ADCC in vitro, it can be seen that both NK92-CD16 and PBMC, as killer/effector cells, can be effectively induced by cetuximab to activate a suicide switch element, thus causing cell death and increasing cell controllability.

The above experimental results show that the killing effect of the ROBO1-iCaspase9 CAR-NK or ROBO1-EGFRt CAR-NK cells of the present invention is equivalent to that of the ROBO1 CAR-NK cells, and the specific killing effect is significantly enhanced compared with the NK92 cells, which can effectively target tumor cells for killing; secondly, the CAR-NK cells have less cytotoxicity, shorter action time and faster killing effect than the CAR-T cells; and finally, because the suicide gene (such as iCaspase9 or EGFRt induced suicide switch element) are also added in the CAR-NK cells of the present invention, the safety and controllability of the CAR-NK cells can be further improved, so that clinical medication is safer and more controllable.

The description of the present invention is given for the purpose of illustration and description, and is not intended to be exhaustive or to limit the present invention to the form disclosed. Many modifications and variations will be apparent to those of ordinary skills in the art. The embodiments are chosen and described in order to better explain the principles and practical applications of the present invention, and to enable those of ordinary skills in the art to understand the present invention, thus designing various embodiments with various modifications suitable for specific uses.

What is claimed is:

1. A nucleotide sequence carrying a suicide gene, comprising a gene encoding a chimeric antigen receptor, and further comprising a suicide inducing gene, wherein the chimeric antigen receptor comprises an antigen binding domain, a transmembrane domain and a costimulatory signal transduction region, the antigen binding domain is capable of specifically binding to a tumor specific antigen, and activating a NK cell through the transmembrane domain and the costimulatory signal transduction region, and the tumor specific antigen is ROBO1.

2. The nucleotide sequence carrying the suicide gene of claim 1, wherein the suicide inducing gene is iCaspase9, EGFRt, CD20, rapamycin and/or RQR8.

3. The nucleotide sequence carrying the suicide gene of claim 2, wherein the suicide inducing gene is iCaspase9, the iCaspase9 comprises FKBP12-F36V and ΔCaspase9, a nucleotide sequence of the iCaspase9 is shown in SEQ ID NO: 6, a nucleotide sequence of the FKBP12-F36V is shown in SEQ ID NO: 1, and a nucleotide sequence of the ΔCaspase9 is shown in SEQ ID NO: 2.

4. The nucleotide sequence carrying the suicide gene of claim 3, wherein the iCaspase9 is further provided with a flag gene, and the flag gene is CD19, Myc, Flag, HA or His.

5. The nucleotide sequence carrying the suicide gene of claim 4, wherein the flag gene is CD19, the iCaspase9 is further provided with a splicing gene, and the splicing gene is T2A; and a nucleotide sequence of the CD19 is shown in SEQ ID NO: 4, and a nucleotide sequence of the T2A is shown in SEQ ID NO: 5.

6. The nucleotide sequence carrying the suicide gene of claim 2, wherein the suicide inducing gene is EGFRt, and the EGFRt comprises an extracellular domain III, an extracellular domain IV and a transmembrane region of EGFR; and a nucleotide sequence of the EGFRt is shown in SEQ ID NO: 12.

7. The nucleotide sequence carrying the suicide gene of claim 1, wherein the antigen binding domain is capable of specifically binding to one or more of Ig1, Ig2, Ig3, Ig4, Ig5, FN1, FN2 and FN3 domains of the tumor specific antigen ROBO1;
wherein the transmembrane domain is selected from one or more of CD28, CD3c, CD45, CD4, CD5, CD8, CD9, CD16, CD22, CD33, CD37, CD134, CD137, ICOS and CD154; and/or,
the costimulatory signal transduction region contains an intracellular domain of a costimulatory molecule, and the costimulatory molecule is selected from one or more of CD3ζ, CD3γ, CD3δ, CD3ε, CD5, CD22, CD79a, CD79b, CD66d, CD2, CD4, CD5, CD28, CD134, CD137, ICOS, CD154, 4-1BB and OX40.

8. The nucleotide sequence carrying the suicide gene of claim 7, wherein the antigen binding domain is capable of specifically binding to the FN3 domain of the tumor specific antigen ROBO1; and the antigen binding domain is an antibody or an antigen binding fragment thereof specifically binding to the FN3 domain of ROBO1, and the antigen binding fragment is Fab or ScFV.

9. The nucleotide sequence carrying the suicide gene of claim 8, wherein the chimeric antigen receptor comprises a fusion protein with a structure of ScFV-CD8-4-1BB-CD3ζ, and the ScFv is capable of specifically binding to the FN3 domain of the tumor specific antigen ROBO1;
wherein an amino acid sequence of the fusion protein ScFv-CD8-4-1BB-CD3ζ is shown in SEQ ID NO: 8 or SEQ ID NO: 9;
wherein an encoding nucleotide sequence of the fusion protein ScFv-CD8-4-1BB-CD3ζ is shown in SEQ ID NO: 10 or SEQ ID NO: 11.

10. A construct carrying a suicide gene, comprising the nucleotide sequence of claim 1.

11. The construct of claim 10, wherein when the suicide gene is iCaspase9, a nucleotide sequence of the construct is shown in SEQ ID NO: 7; or
when the suicide gene is EGFRt, the nucleotide sequence of the construct is shown in SEQ ID NO: 13.

12. A chimeric antigen receptor carrying a suicide gene, encoded by the nucleotide sequence of claim 1.

13. A ROBO1 CAR-NK cell carrying a suicide gene, wherein the cell expresses the chimeric antigen receptor of claim 12; and the ROBO1 CAR-NK cell carrying the suicide gene is capable of effectively destroying or killing a lung cancer cell, a pancreatic cancer cell, a hepatoma cell, a breast cancer cell, a colon cancer cell, a prostate cancer cell or a gastric cancer cell.

14. A preparation method of the ROBO1 CAR-NK cell carrying the suicide gene of claim 13, comprising the following steps of:
(1) synthesizing and amplifying the suicide gene in the nucleotide sequence carrying the suicide gene of claim 1, and cloning the nucleotide sequence into a lentiviral expression vector to obtain a lentiviral vector carrying the suicide gene; and
(2) packaging a lentivirus through a lentivirus packaging cell line and a three-plasmid system comprising the lentivirus vector carrying the suicide gene obtained in step (1) to obtain a lentivirus carrying the suicide gene, and then infecting the ROBO1 CAR-NK cell with the lentiviral carrying the suicide gene to integrate the suicide gene into a genome of the ROBO1 CAR-NK cell to obtain the ROBO1 CAR-NK cell carrying the suicide gene.

15. The preparation method of the ROBO1 CAR-NK cell carrying the suicide gene of claim 14, wherein the ROBO1CAR-NK cell is prepared through a method comprising the following steps of:
a. synthesizing and amplifying a nucleotide sequence encoding the chimeric antigen receptor, and cloning the nucleotide sequence into a lentiviral expression vector; and preferably, synthesizing the encoding nucleotide sequence of the fusion protein ScFv-CD8-4-1BB-CD3ζ in claim 9 to obtain a lentiviral vector containing the encoding nucleotide sequence of the fusion protein ScFv-CD8-4-1BB-CD3ζ;
b. packaging through a lentiviral packaging plasmid and the lentivirus expression vector obtained in step a in a packaging cell line to prepare a lentivirus; and
c. infecting a NK cell with the lentivirus obtained in step b to obtain the ROBO1 CAR-NK cell.

16. A pharmaceutical composition, comprising the ROBO1 CAR-NK cell carrying the suicide gene of claim 13;
and the pharmaceutical composition further comprising an inducer, wherein when the suicide gene is iCaspase9, the inducer is AP1903 or AP20187; and a concentration of the inducer is 0 nM to 50 nM; or
when the suicide gene is EGFRt, the inducer is cetuximab with an action concentration of 1 μg/ml; and
an effector-to-target ratio of the ROBO1 CAR-NK cell carrying the suicide gene to a tumour cell is 0.5:1 to 5:1.

17. A method for preparing a medicament for treating and/or preventing a cancer, comprising using the ROBO1 CAR-NK cell carrying the suicide gene of claim 13, wherein the cancer is a tumor with high expression of ROBO1 and related diseases.

18. The method of claim 17, wherein the medicament is a medicament in an intratumoral administration form, such as a medicament in an intratumoral injection form or a medicament in an intravenous infusion form.

19. A method for treating and/or preventing a cancer by using the ROBO1 CAR-NK cell carrying the suicide gene of claim 13, wherein the method comprises administering an effective amount of the pharmaceutical composition containing the ROBO1 CAR-NK cell carrying the suicide gene into a patient.

20. The method of any one of claim 19, wherein a dosage of the ROBO1 CAR-NK cell carrying the suicide gene is 0.5×10⁹ cells/times to 5×10⁹ cells/times.
